SEQUENCE LISTING (146 sequences)

SEQ ID NO: 1 - DNA, 463 bp, Homo sapiens
cgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcaaaaaaaaaaaaaaaaaaaaaaaaaa

SEQ ID NO: 2 - DNA, 313 bp, Homo sapiens
ggccaatcagattttctatgttctaaggacatggctgctgtagaatagcacagacgtggatgataaattatccccagaagcagcatgacagaatgcctcggggagcacttggaagggaaattgcagttctgttgaaatagaggaaaatcccttggtaaggacacagcctgttaggctcgtgtgggcctccagtatgttcaccaggggaatggctgggatttctcggcactctgcatcatccatcttttcttataggtgggaaaataaacaactttgtgatcctcctgaaaaaaaaaaaaaaaaaaaaaaaaaa

SEQ ID NO: 3 - DNA, 225 bp, Homo sapiens
ttttttttttttttttttttttttttgccttttaaaacagcagctttaatgccccccagaatcagcaccatgtcatcacaggcttgggtcaaggggcgggtcagacgccagtcacatccgctcactgcccacagccacccccccacagtgagtcatctgccagggccgctccagagtctatacacaggggtggtctcttcaataaagaagtgttgattagacctg

SEQ ID NO: 4 - DNA, 511 bp, Homo sapiens
ttttttttttttttttttttttttttgatttaataaagttttatttttccaaatgtacagctggttggacctattcatgcatcttcaccagcagctggagcatctccacccttggtatttctggtgtaaattacttgagctctgtgctttgaaaccagtttgataagtcctttactaaggagctcctgaagggctgccctggccagggagcctcgaatcttcagtctctcagagaccacagctggggttataagtttatagttgggaacttccttacagagtttatcataggtagctttgtcaaacaagactaagttattgagcttgtcccgaactttgcctttggaccacttcttctttttggccttgcccccggatttgttcactgggtctttgtctttcttggccgactttccagcgtccttcttcttcttgtcgtccttaggcggcattgcgaagctcggagaatagcagcagacaccgcagcctcgtcaagatgtcggacaaaaag

SEQ ID NO: 5 - DNA, 501 bp, Homo sapiens
ctcttttccggttagcgcggcgtgagaagccatgagcagcaaagtctctcgcgacaccctgtacgaggcggtgcgggaagtcctgcacgggaaccagcgcaagcgccgcaagttcctggagacggtggagttgcagatcagcttgaagaactatgatccccagaaggacaagcgcttctcgggcaccgtcaggcttaagtccactccccgccctaagttctctgtgtgtgtcctgggggaccagcagcactgtgacgaggctaaggccgtggatatcccccacatggacatcgaggcgctgaaaaaactcaacaagaataaaaaactggtcaagaagctggccaagaagtatgatgcgtttttggcctcagagtctctgatcaagcagattccacgaatcctcggcccaggtttaaataaggcaggaaagttcccttccctgctcacacacaacgaaaacatggtggccaaagtggatgaggtgaagtccacaatcaagtt

SEQ ID NO: 6 - DNA, 331 bp, Homo sapiens
ttttttttttttttttttttttttttaattcttcagctaaaacagcggaagaggtgatttattatatggttgttacactcggccacaaataaacacagaaatagtccagaatgtcacaggtccagggcagaggaccaacatgggcattttgtttatgagcaaggtgggtctcagaggtgatcggcgatcagagggcgatgaagttctagatccattgagacaagctctagacagtagcatgcagtcccacaacttgtaccagcatccccagcgtctggcattccatgtttctgctcctgtggcctccacggtgcaacaagctagcgggggg

SEQ ID NO: 7 - DNA, 320 bp, Homo sapiens
cctttggaagagagaagaacagagtgttcgattctgccctatttatgtttctactcgggaacaaacgttggttgtgtgtgtgtgtgttttcttgtgttggttttttaaagaaatgggaagaagaaaaaaaaattctccgcccctttcctcgatctcgctccccccttcggttctttcgaccggtcccccctcccttttttgttctgttttgttttgttttgctacgagtccacattcctgtttgtaatccttggttcgcccggttttctgttttcagtaaagtctcgttacgccaaaaaaaaaaaaaaaaaaaaaaaaaa

SEQ ID NO: 8 - DNA, 333 bp, Homo sapiens
ttttttttttttttttttttttttttcatggtcgaaataattttatttatccagaatatacagtttaattcctctatctacacttatttacatggctaaaataacattgaaaaaagtcttttgaaaagttgaggtcatagatttcaaggcaccattgaaagtgtccacagttgcgcaaaaaaagtcctctgtaaaaaaagggggggtcttttgaaatgcaataacttacatgcaaaaaaaagctttacacatgaatctttttcagttttccgaacttccccatatgaattcctttctttatgattttctagaacagcaaaacacagtagtccc

SEQ ID NO: 9 - DNA, 440 bp, Homo sapiens
ttttttttttttttttttttttttttgaaatttaaagaaaaaatttattgaagatctgaaaaacaattcctaaaagattgacttttccagaaaactagctacacaatgcattgcgtctatcatgttaaaacgtgcattagacacaaatacaaaaaccatgaaacaagccaccattcttcaacaatttgagccaagataaaatgcctaagtaacaacatggatgacttgcaaaggatgggctctttactttaagcaccataaaaaaaaaacaaaagcacaaatggatgagtgtgttcagttatatacactgaattgaacctttggcactaggaatcagagcattttgtcatatagcattaacatgtattataaaagtgcgtagtgtcaaaggaatagaaccaccagcattcaaaagcagctttgtcaactaggcaataaaa

SEQ ID NO: 10 - DNA, 369 bp, Homo sapiens
gggaaagtaacacatgaagtagttcagtcatgtcaggtttgtctggggtggaatggaacagtcaggtagttgaaagtttttttttagagatgaaaagcttgtgaactcctgtaaaacatgctgtatttgaaatacatctgttaaaacttaaaaactaaagtgtgattttttttgttgttgttaagtttattgaagtttattcccttaatcagtgaaggacaacctttatttattacctagagcagttgtataattcgctgttagaaattttggtattgggacagtggataagcaggttatcacatagcatagaattcttaaaagatacatgtggggaaaagtagtctcaaataaaagctaatttctttg

SEQ ID NO: 11 - DNA, 234 bp, Homo sapiens
ttttttttttttttttttttttttttgaagcttctcacatctttattggaaaggcacagctaagcccacccttgatacagcattttccacttctctctgtggagatcagacgattgaacacgagataagctgactatatacagacacaggtgtgggtgttgcttttttttaaaaacacttttgtcttttttttttttaatatcccctttcttaaaagacaagctagtatactgg

SEQ ID NO: 12 - DNA, 237 bp, Homo sapiens
ttttttttttttttttttttttttttgctcgggtttattttccaaacaattttattgaaatgtgccaagagtacatgggcagcacaaatgtatgaacaggaaaaaaaaaaatcacatgtacaataattttttaaaagtgaaggttaatcttgggagataacagttcccctctccctccccctgcagatttccagcgtttccattaaggttaagtaagcctctacaaacctagcattc

SEQ ID NO: 13 - DNA, 302 bp, Homo sapiens
cttttgtaaaggcaatgagctagtcccagaaaggatccttcagttacatacaatttgtttaatgaaatgtcatggctctgttcatacttttgtcttgttcttccaattggtatatacaactttcagagcctcttgtatttggaaggctggaagggcccacactttggaatagcgtcttggtttcactgtttttgctttgattttttttttgttttgattttttttaaactaaagccatataaagcttgtggattaaacagaataaatttctaaatttaaaaaaaaaaaaaaaaaaaaaaaaa

SEQ ID NO: 14 - DNA, 410 bp, Homo sapiens
ccctcttgtgctcagtcgccctgttcttgaggtctcttttctctactccatggttctcaatttatttggggggaaataccttgagcagaatacaatgggaaaagagtctctacccctttctgttcgaagttcatttttatcccttcctgtctgaacaaaaactgtatggaatcaacaccaccgagctctgtgggaaaaaagaaaaacctgctcccttcgctctgctggaagctggagggtgctaggcccctgtgtagtagtgcatagaattctagcttttttcctcctttctctgtatattgggctcagagagtacactgtgtctctatgtgaatatggacagttagcatttaccaacatgtatctgtctactttctcttgttcaaaaaaaaaaaaaaaaaaaaaaaaaa

SEQ ID NO: 15 - DNA, 220 bp, Homo sapiens
gaaaagtcacaattttataaaaatggtttttcttacattcttttgagaactgtttcactcatacatacacccacacaccccactcaaccttgtatcaaattccaaaagtgtaactaaagtataagaatatcatgactagttaaaagatagcaaataccataaggtacaagttcaagtattagtataacaagtatctgagtaacaaatgtccttggaaatg

SEQ ID NO: 16 - DNA, 223 bp, Homo sapiens
ttttttttttttttttttttttttttgactaaaggacatttatttatttttaccaagactttatcttgaggacatggctaaactgcatttccaccccccaccccatcttgaagaagggttagtctgtgaatgttataaagtagatatgaacagttgaaggactggaaccaacattaagtgacgaagaacaacttccatctaaatcatcataaaaatgtttaag

SEQ ID NO: 17 - DNA, 397 bp, Homo sapiens
gggtgaggggaccctactctgttatcccaagtgctcttattctggtgagaagaaccttacttccataatttgggaaggaatggaagatggacaccaccggacaccaccagacactaggatgggatggatggttttttgggggatgggctgggggaaataaggcttgctgtttgttctcctggggcgctccctccaacttttgcagattcttgcaacctcctcctgagccgggatcgtccaattactaaaatgtaaataatcacgtattgtggggaggggagttccaagtgtgccctcctctcttctcctgcctggattatttaaaaagccatgtgtggaaacccactatttaataaaagtaatagaatcagaaaaaaaaaaaaaaaaaaaaaaaaaa

SEQ ID NO: 18 - DNA, 319 bp, Homo sapiens
tccttggaaatgctgcgctctttgtgtttttccatcattagtgcagttggaatgaatgtgtataggtcagaggtcttcgtgttcacattttaaaattaggtaaatgacctcatctttcaagcttgaattcatttttaattttaattttattttatacaatgtgtagacagttccctgttctctgcatttagaagtatacacaatataaatctgttaattctgtaagtaatttttataattatgatataactctatcttatccttaaaacatttaaaataaaccctttatgtgcaaaaaaaaaaaaaaaaaaaaaaaaaa

SEQ ID NO: 19 - DNA, 190 bp, Homo sapiens
aactaggcacatttttttaatgagctacaaactaaattacaaataacgttaatagcaggagcactcaaggacaagggtgacagagacaaatcataaaaaaaaaatgcttttttttttagcactgtcattacaaaaccccaaggtgcacatggcaacaaatccacccactatgaccaaaaaaaaaaaaaaa

SEQ ID NO: 20 - DNA, 261 bp, Homo sapiens
gttataatctattcatggatctccactttgatttaatttttaaatgttttaatctcctttacaaaaagtatacgttaatataccgtcctcaagggggaactggcactgggaccttagcatttagttttctagaggatgtgatctaatttctttctagctcatcattaaaaaggaaattgtatcaggacccatgggatatatccagaggcaaactttatgaggctttgaaatcttgccttcctgaagatagctgagtgggag

SEQ ID NO: 21 - DNA, 77 bp, Homo sapiens
ttttttttttttttttttttttttttcacactcgccgtctttattttgcactcaccctgggtgacactgggcaggcc

SEQ ID NO: 22 - DNA, 401 bp, Homo sapiens
cgaaaacacatgtaattttatttttgtttaaccttaagcttgccaacttctctccctgaacagcatttgtcttgttttgatacccacctacacttatattagaaacgtactgcaaactatttagtgactccacttttaatttatggtcgtatgactaagatgttttgaatataagcctatcggtatgccagatataaatttcaggaactcttaatgtctcttcttttacagaaactctacctctatcctgatgtaattaggttgattaagaaaattaaaggccacgggccagaaaactaacttccacaaatgcaacaaaaggaaaatttctccaataacctgtatgcaatgggaaatctttcttttaagtaccaagtcaatttctttaactgtacaatgat

SEQ ID NO: 23 - DNA, 408 bp, Homo sapiens
gagacaacgtctggctctgtcacccaggctggagcgcagtggcacaatctcggcccactgcagcctcgacctctggggctcaagggatcctcatgcctcagcctcatatgtagctggggccacagacatgcaccaccacacctggctcgaggccattttagttctgaggttgagcagatcaggatccggctccagcacggtgctgtgtttgtgaaacacagaaaggggacccccgaggaccccagacagggccttaggactctcatatcttacttgtcttctccatctggtggctcttgctctctggttttctttaccttttcatggccccagaatccatatgcagtaaaggaactctctggaatataattaaagtcctcccaaaaaaaaaaaaaaaaaaaaaaaaaa

SEQ ID NO: 24 - DNA, 232 bp, Homo sapiens
cagtggaaaataacttttattgagaccccctcaggattgtttctcggcaccagtgcctcagtgctgcaattgagggctaaatcccaagtgtctcttgactggctcaagaattagggccccaactacacacccccaagccacagggaagcatgtactgtacttcccaattgccacattttaaataaagacaaatttttatttcttctaaaaaaaaaaaaaaaaaaaaaaaaaa

SEQ ID NO: 25 - DNA, 129 bp, Homo sapiens
ttttttttttttttttttttttttttcagaacattcacctttaattctgttacaggttccagtatttgcgctggccttagtcagctgtgggtaagagtcacccatgggcaagagtggggaggggactgc

SEQ ID NO: 26 - DNA, 178 bp, Homo sapiens
acgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtctcaaaaaaaaaaaaa

SEQ ID NO: 27 - DNA, 127 bp, Homo sapiens
ccatccaatccacacccagcagacccttcggcatgccgccctctaccaggaagccagaggcctaggagctcgccatccatatttatttgaaaaggtcaaaaggagcatctatgagacaagggagggg

SEQ ID NO: 28 - DNA, 264 bp, Homo sapiens
caggtcgtttttaaagtatttctttttttaactggatgaaaaatcttcatgttaggattaattttcttaatcacctccacactgtacagaggaaactcaagccttaaatgtttaagtaaactctgtctcagttttaggattaaaatacccaccggtggtgtgatgatgccatataccgcagggcttgcttctgtcaagtgtgactctatctcagtaattaaaataagtgctgctctacaaaaaaaaaaaaaaaaaaaaaaaaaa

SEQ ID NO: 29 - DNA, 391 bp, Homo sapiens
gagacagcgtctcactcccttgcccaggttggaatgcagtggcgtgatcttggcccactgcaacctccatctcccatgttcaagtaattctcctacctcagcctcccaagtagctgggattacaggcatgcaccaccacgcccagctaatttttgtattcctagtagaaacagggtttcaccatgttggccaggctggtctcgaactcctgacctcaagtgacccgcctgcctcggcctcccaaagtgctgagattactggcgtgagccaccatgcctggctgataggaatatttttgaaagaccttttcatggtactttacatcctgaaaggaatggctttttcttctctcaggagaacatgtaagattaacaagtatttagactaaagc

SEQ ID NO: 30 - DNA, 379 bp, Homo sapiens
gggcttttcataccacacacatatatttcctatttctcaggaatggtgctgtgtcattactatgcacaaaatcggattcttcaagaaaattgttttttaggccgagcatggtggctcacgcctgtaatcccagcactttgggaggccatggcaggtggatcacgaggtcagtagttcgagaccagcctggccaacatggtgaaatcccatctctactaaaaatacaaaaattagccgagcgtggtggcacacgcgtgtaatcccagctactcagaaggctgaggcagaagaatcgcttgaacctgggagacggaggttgcagtgagccgagatcgcaccattgcactccagcctgggtgacagggcaagactccatctc

SEQ ID NO: 31 - DNA, 196 bp, Homo sapiens
gcactgtgtgacatgctttaatatatatattttttatgtatccaaagaggtaatcaactttaactttcagaattcatgaatcttggtttggtggcatgaatgaaaggttatgtaaccccaccagtaactcatttggggtgcccttgaaatattaaagtttggtcctcaataaaaaaaaaaaaaaaaaaaaaaaaaa

SEQ ID NO: 32 - DNA, 473 bp, Homo sapiens
ttttttttttttttttttttttttttcaagaattgacacccaagaccatcctttattgtagtattagttcatggtaactgcatgaaaaaacatttcaggaggaatttacaatttccagcttaaagaacttgcccaccaacataaccaatttatgaaagtcaattcattaaaaggtatagaacctcttgttgggcatgatggcaagggacaaagctacaacttggcctgtgcctttggaagctgaggcaggaggaccatctgagcccaggagcctgagaccagcctgggcaacatagagaatccgtctcaacaaaaaaaaaattttagccaggtgtgctgtgagctgtagtcccagctacaaggtgggaggattgcttaggcctgggtgattgaggatgcaatgagctgtgattgtgccaccacactccagcctgggcaatacagcaagactgtctcaaaaaaaaaaaaaaaaa

SEQ ID NO: 33 - DNA, 493 bp, Homo sapiens
ttttttttttttttttttttttttttaaaatcacaaataatttattcttccgttagagttgatacagttttaaatagcaacaaagtacacagtggtggaaaattggcacagaacctacaagcatttcaatcacaataaggctgtccatgatttatctgttcaaacttgatgtccattttctactgatcaagttgaaagaaaaaacctattctggccaggcacggtggctcacgcctgtaatcccagcactttggaaggccgaggcgggtggatcacctgaggtcaggagttcgagaccagcctggccaacatggcgaaacctcgtctctactaaaagtacaaaaattagctgggtgtggtggcgggtgcctgtcatcccagctactcaggaggctgaggcaggagaactgcttgaacctgggaggcggaggttgcagtgagctgagattgcgccaccgactccagcctgggcgacaagagcaagactccatcc

SEQ ID NO: 34 - DNA, 207 bp, Homo sapiens
ttttttttttttttttttttttttttctcacaaactggttgcatttgttgatttacttttccatccttttcaccagtaaagattatcatcgtttatttttgtttttaaagtcgaaaacaaaaaagaggagagaacagagttatggtatgaatgtatgtaaaactatagagaactacagtaatatgtactgtggttattgctgtcttc

SEQ ID NO: 35 - DNA, 195 bp, Homo sapiens
ttttttttttttttttttttttttttgtggctcaataaacttttattgttttgttatttatttttattcaaaagaatttgtttccctatcacaaaaattagataacaccaccaagagcagcaatgtccatggagttacattgatggtggagagttggctgggtattcccaatgtcccaaaggtcataaaggaggg

SEQ ID NO: 36 - DNA, 373 bp, Homo sapiens
caaattccaaccagaagctaaatacaattggaaactggtaagcactagttttactccaaaggagtaggatcattcagatttactccaataaaagtatgcaacccttaagcaaagctttttttcatttaaatggagaaaaaaaacaacctatacagtagtctttccttatgttcattgcacaaaatgagttctgcttttagaactttgacactcaatggttaattttacaatttaagattccaactttataaccttttttctactccaaaacacccttgtaaagtttttctttaggatggtgtaaaaaccagcatttctgcacaattcactggaatttttttctttgtaataaaaatctcttctctgtaaaacc

SEQ ID NO: 37 - DNA, 246 bp, Homo sapiens
gctggccatgagaattagacttcatattagcttcaattaaaaagctcattccattttattaaaatgtttgtaattgcaattttatgtttaatgtttactttgtccttcctttctttagcatttaggtgactgttcggcagtttgatatatggccattgtcggccttaaactgcactagtaacaagcatggtatttatcttgtattgaaaataaaacaaacttttgagaaaaaaaaaaaaaaaaaaa

SEQ ID NO: 38 - DNA, 507 bp, Homo sapiens
gagacggagtcttgctctgttgcccaggcaggagtgcagtggcgcatctcggctcactgcaacctctgtctcaagcgattctcctgcctcagcctcctgagtagctgggattacaggtgcatgccaccaggccccgctgatttttgtatttttagtagagatagggtttcaccatgttggtcaggctggtcgtgaactcctgacctcatgatctgcccgcctcaacctcccaaagtgctgggattataggcgtgagccaccgcccccagctgattccaggcgaattcttctctgatgggcgggcgagggtgtgttcgtgtgatgggttggagtgtgtgtgtctgagtacacagatgatgtgttttcccttcagcttcttacgttttctgagcatccgttgtgccttaacattttctgcttgtcctttgggacaaagcagtattttactcattctttgaatgttctcattcttttgtatcatgtgacttattaaaatcagtttctaac

SEQ ID NO: 39 - DNA, 217 bp, Homo sapiens
gggcaggcccatctcagaattaacgctttgatggcatcaccgcgtcgggaatccctggggatggcgttctccaccgtcaagacctttgagccgcctgagcgactaactcctgcgcccctgaggtcacagctacttggaaggctgagatgtgaagactacttgagctcagaagttccactgcactccagcctgggttacagagggaaaccttgtctct

SEQ ID NO: 40 - DNA, 204 bp, Homo sapiens
cataagaaaactacgttaattttaccttactttcattgtaaataagcctgtcttcctatctggattttttgtgtgcatacatattctactgattaactacttttgcagctttaatcctgtattatttcttctactttgttttgtgtaaaaggggaaaaaataaaaaagctggaatcttcaaaaaaaaaaaaaaaaaaaaaaaaa

SEQ ID NO: 41 - DNA, 257 bp, Homo sapiens
ttttttttttttttttttttttttttgaagcttttatcaactttaattgatataattataattggtatgcaatatgtcttacaacaaatgtggatacaatgacatcaaataccaaagaattttcccacctttccatctttaaatgacttcagctaacttaccccaagagaaaaatttggcattgatctcttggttagcattattaccaatatcaactagcactattaaaatcaaagttgaaatggtacattcatttg

SEQ ID NO: 42 - DNA, 322 bp, Homo sapiens
gtaattatatgaagaaagtccagttctcataaatattgatcacttaaaaaacttactctttcttgaagaggtacacatgtaaaatttaggaaaatagctaaagtaggggctggaaccataagaatgtttatccgcacgttcatttattattttggatttggaacttggctttgtttttcaatagtgacaagaatggttcagttctaggaatgttctggaagatgctgctaattttactttaaaatgagaatctggtgttactgtattttatcgttttcaataaaacttcttaagtgttttggaaaaaaaaaaaaaaaaaaaa

SEQ ID NO: 43 - DNA, 491 bp, Homo sapiens
gccactgcactccagcctgggtgacacagcaagaccttgtccctaaataattaaataaatacatctgtgctatctcatactagagctactagtcacatggggttactgagcagtggaaatgtggctagtctaaattgagatatgctctacatgcataatatacttcagattttaaagacttggtatgaaacaaagaatgtaaaatatctccttaatagtttttatattgattataagttgaaatattttggatacattagattaaataataaagtgagaattaactttacttgctttttacctttttaatgtagctaaaagaaaatgtgaaataaaatgtgtggcttatgtttctattaaaccatgctgttttaaataaaaaatccataatgctcagtatagatacagtgattaacacaaaagtataggctgaaagagcttaggaatatttctcactgggaacgtggtgtataaatttatattaagaatat

SEQ ID NO: 44 - DNA, 367 bp, Homo sapiens
cagggatcagatttttatttatctttatccccagcacttgatgtggcacttggtatacagtaagtactcagtaaagatttggatggatgggccgggcacagtggctcaagcctgtaatcccagcactatgggaggccgaggtgggaggatcacaaagtcaggagtttgagaccaccctggccaatatggtgaaaccctgtctctactgaaatacaaaaattagccaggcatggtggtgggcgcctgtagttccagctactccggaggctgaggcaggagaatcacttgaacccgagaggcggaggttgcagtaagccgagattgtgccaccgaactccacctgggtgagaaagcgagactccatctc

SEQ ID NO: 45 - DNA, 342 bp, Homo sapiens
caaagttgacagataacatttattaaaacatgtcatacaaaagggcatgatctcttctataagaagaaaatattaaacattaacattcaattaagtaaaaccatgctgcacactgaagacagcaatatataaagacaaaactattcaacttttgcaacataggcataacatttcacaaaatatcaataagatatgcacctaatgataatctgtttatcatcaaatggttttgtggcctaagagtcacctgacttgcataaataaagtacttttctggacgaaacattgggaacctcacaaaaggaaacaaggcagcagcacctaacatgacacaggcttcaa

SEQ ID NO: 46 - DNA, 21 bp, Artificial
  synthetic pcr primer
gtgtaacaattgtgtagcctt

SEQ ID NO: 47 - DNA, 20 bp, Artificial
  synthetic pcr primer
ggcaactggtaaactgtcca

SEQ ID NO: 48 - DNA, 20 bp, Artificial
  synthetic pcr primer
ggccaatcagattttctatg

SEQ ID NO: 49 - DNA, 20 bp, Artificial
  synthetic pcr primer
ggaggatcacaaagttgttt

SEQ ID NO: 50 - DNA, 18 bp, Artificial
  synthetic pcr primer
ggtggctgagggcagtga

SEQ ID NO: 51 - DNA, 22 bp, Artificial
  synthetic pcr primer
gccttttaaaacagcagcttta

SEQ ID NO: 52 - DNA, 20 bp, Artificial
  synthetic pcr primer
ctgcggtgtctgctgctatt

SEQ ID NO: 53 - DNA, 20 bp, Artificial
  synthetic pcr primer
tgagcttgtcccgaactttg

SEQ ID NO: 54 - DNA, 20 bp, Artificial
  synthetic pcr primer
ttggcctcagagtctctgat

SEQ ID NO: 55 - DNA, 20 bp, Artificial
  synthetic pcr primer
tgtggacttcacctcatcca

SEQ ID NO: 56 - DNA, 20 bp, Artificial
  synthetic pcr primer
gaacttcatcgccctctgat

SEQ ID NO: 57 - DNA, 20 bp, Artificial
  synthetic pcr primer
gttgttacactcggccacaa

SEQ ID NO: 58 - DNA, 22 bp, Artificial
  synthetic pcr primer
gagagaagaacagagtgttcga

SEQ ID NO: 59 - DNA, 20 bp, Artificial
  synthetic pcr primer
caggaatgtggactcgtagc

SEQ ID NO: 60 - DNA, 21 bp, Artificial
  synthetic pcr primer
ggaagttcggaaaactgaaaa

SEQ ID NO: 61 - DNA, 21 bp, Artificial
  synthetic pcr primer
tcatagatttcaaggcaccat

SEQ ID NO: 62 - DNA, 20 bp, Artificial
  synthetic pcr primer
gcccatcctttgcaagtcat

SEQ ID NO: 63 - DNA, 21 bp, Artificial
  synthetic pcr primer
cacaatgcattgcgtctatca

SEQ ID NO: 64 - DNA, 20 bp, Artificial
  synthetic pcr primer
gggaaagtaacacatgaagt

SEQ ID NO: 65 - DNA, 20 bp, Artificial
  synthetic pcr primer
cagcatgttttacaggagtt

SEQ ID NO: 66 - DNA, 19 bp, Artificial
  synthetic pcr primer
gcaacacccacacctgtgt

SEQ ID NO: 67 - DNA, 22 bp, Artificial
  synthetic pcr primer
gcttctcacatctttattggaa

SEQ ID NO: 68 - DNA, 20 bp, Artificial
  synthetic pcr primer
gagtacatgggcagcacaaa

SEQ ID NO: 69 - DNA, 21 bp, Artificial
  synthetic pcr primer
ccttaatggaaacgctggaaa

SEQ ID NO: 70 - DNA, 20 bp, Artificial
  synthetic pcr primer
gcaatgagctagtcccagaa

SEQ ID NO: 71 - DNA, 20 bp, Artificial
  synthetic pcr primer
cccttccagccttccaaata

SEQ ID NO: 72 - DNA, 21 bp, Artificial
  synthetic pcr primer
tatcccttcctgtctgaacaa

SEQ ID NO: 73 - DNA, 19 bp, Artificial
  synthetic pcr primer
gcctagcaccctccagctt

SEQ ID NO: 74 - DNA, 21 bp, Artificial
  synthetic pcr primer
actcatacatacacccacaca

SEQ ID NO: 75 - DNA, 22 bp, Artificial
  synthetic pcr primer
acttgaacttgtaccttatggt

SEQ ID NO: 76 - DNA, 22 bp, Artificial
  synthetic pcr primer
cttaatgttggttccagtcctt

SEQ ID NO: 77 - DNA, 22 bp, Artificial
  synthetic pcr primer
caagactttatcttgaggacat

SEQ ID NO: 78 - DNA, 25 bp, Artificial
  synthetic pcr primer
gaaataaggcttgctgtttgttctc

SEQ ID NO: 79 - DNA, 18 bp, Artificial
  synthetic pcr primer
ccggctcaggaggaggtt

SEQ ID NO: 80 - DNA, 20 bp, Artificial
  synthetic pcr primer
cttggaaatgctgcgctctt

SEQ ID NO: 81 - DNA, 22 bp, Artificial
  synthetic pcr primer
aagcttgaaagatgaggtcatt

SEQ ID NO: 82 - DNA, 20 bp, Artificial
  synthetic pcr primer
gggtggatttgttgccatgt

SEQ ID NO: 83 - DNA, 26 bp, Artificial
  synthetic pcr primer
gctacaaactaaattacaaataacgt

SEQ ID NO: 84 - DNA, 22 bp, Artificial
  synthetic pcr primer
tattcatggatctccactttga

SEQ ID NO: 85 - DNA, 22 bp, Artificial
  synthetic pcr primer
gatcacatcctctagaaaacta

SEQ ID NO: 86 - DNA, 19 bp, Artificial
  synthetic pcr primer
gcactcaccctgggtgaca

SEQ ID NO: 87 - DNA, 19 bp, Artificial
  synthetic pcr primer
gtcccctgctcgggagtaa

SEQ ID NO: 88 - DNA, 20 bp, Artificial
  synthetic pcr primer
ctggcataccgataggctta

SEQ ID NO: 89 - DNA, 21 bp, Artificial
  synthetic pcr primer
gttttgatacccacctacact

SEQ ID NO: 90 - DNA, 19 bp, Artificial
  synthetic pcr primer
gacagggccttaggactct

SEQ ID NO: 91 - DNA, 21 bp, Artificial
  synthetic pcr primer
cagagagttcctttactgcat

SEQ ID NO: 92 - DNA, 20 bp, Artificial
  synthetic pcr primer
gattgtttctcggcaccagt

SEQ ID NO: 93 - DNA, 21 bp, Artificial
  synthetic pcr primer
gtacagtacatgcttccctgt

SEQ ID NO: 94 - DNA, 24 bp, Artificial
  synthetic pcr primer
cagaacattcacctttaattctgt

SEQ ID NO: 95 - DNA, 19 bp, Artificial
  synthetic pcr primer
gcagtcccctccccactct

SEQ ID NO: 96 - DNA, 20 bp, Artificial
  synthetic pcr primer
ctgcttcatccgccaactaa

SEQ ID NO: 97 - DNA, 21 bp, Artificial
  synthetic pcr primer
ccacatctacaaaatgccagt

SEQ ID NO: 98 - DNA, 18 bp, Artificial
  synthetic pcr primer
cccctcccttgtctcata

SEQ ID NO: 99 - DNA, 20 bp, Artificial
  synthetic pcr primer
catccaatccacacccagca

SEQ ID NO: 100 - DNA, 20 bp, Artificial
  synthetic pcr primer
ctccacactgtacagaggaa

SEQ ID NO: 101 - DNA, 21 bp, Artificial
  synthetic pcr primer
gagatagagacacacttgaca

SEQ ID NO: 102 - DNA, 20 bp, Artificial
  synthetic pcr primer
gaaagaccttttcatggtca

SEQ ID NO: 103 - DNA, 24 bp, Artificial
  synthetic pcr primer
gctttagtctaaatacttgttaat

SEQ ID NO: 104 - DNA, 20 bp, Artificial
  synthetic pcr primer
catgctcggcctaaaaaaca

SEQ ID NO: 105 - DNA, 21 bp, Artificial
  synthetic pcr primer
gggcttttcataccacacaca

SEQ ID NO: 106 - DNA, 20 bp, Artificial
  synthetic pcr primer
tgtggacttcacctcatcca

SEQ ID NO: 107 - DNA, 22 bp, Artificial
  synthetic pcr primer
gggttacataacctttcattca

SEQ ID NO: 108 - DNA, 20 bp, Artificial
  synthetic pcr primer
caaaggcacaggccaagttg

SEQ ID NO: 109 - DNA, 21 bp, Artificial
  synthetic pcr primer
gaccatcctttattgtagtat

SEQ ID NO: 110 - DNA, 21 bp, Artificial
  synthetic pcr primer
ctttcaacttgatcagtagaa

SEQ ID NO: 111 - DNA, 22 bp, Artificial
  synthetic pcr primer
cttccgttagagttgatacagt

SEQ ID NO: 112 - DNA, 21 bp, Artificial
  synthetic pcr primer
gacagcaataaccacagtaca

SEQ ID NO: 113 - DNA, 21 bp, Artificial
  synthetic pcr primer
ttccatccttttcaccagtaa

SEQ ID NO: 114 - DNA, 20 bp, Artificial
  synthetic pcr primer
ccctcctttatgacctttgg

SEQ ID NO: 115 - DNA, 24 bp, Artificial
  synthetic pcr primer
caaaagaatttgtttccctatcac

SEQ ID NO: 116 - DNA, 22 bp, Artificial
  synthetic pcr primer
gtgtcaaagttctaaaagcaga

SEQ ID NO: 117 - DNA, 21 bp, Artificial
  synthetic pcr primer
ccaaaggagtaggatcattca

SEQ ID NO: 118 - DNA, 24 bp, Artificial
  synthetic pcr primer
gcttcaattaaaaagctcattcca

SEQ ID NO: 119 - DNA, 20 bp, Artificial
  synthetic pcr primer
gtgcagtttaaggccgacaa

SEQ ID NO: 120 - DNA, 20 bp, Artificial
  synthetic pcr primer
gctgattccaggcgaattct

SEQ ID NO: 121 - DNA, 22 bp, Artificial
  synthetic pcr primer
tcagaaaacgtaagaagctgaa

SEQ ID NO: 122 - DNA, 19 bp, Artificial
  synthetic pcr primer
gcatcaccgcgtcgggaat

SEQ ID NO: 123 - DNA, 21 bp, Artificial
  synthetic pcr primer
gagctcaagtagtcttcacat

SEQ ID NO: 124 - DNA, 25 bp, Artificial
  synthetic pcr primer
gaaaactacgttaattttaccttac

SEQ ID NO: 125 - DNA, 24 bp, Artificial
  synthetic pcr primer
caaagtagaagaaataatacagga

SEQ ID NO: 126 - DNA, 22 bp, Artificial
  synthetic pcr primer
ggtaataatgctaaccaagaga

SEQ ID NO: 127 - DNA, 22 bp, Artificial
  synthetic pcr primer
ggtatgcaatatgtcttacaac

SEQ ID NO: 128 - DNA, 20 bp, Artificial
  synthetic pcr primer
gggctggaaccataagaaga

SEQ ID NO: 129 - DNA, 21 bp, Artificial
  synthetic pcr primer
cagtaacaccagattctcatt

SEQ ID NO: 130 - DNA, 23 bp, Artificial
  synthetic pcr primer
ccataatgctcagtatagataca

SEQ ID NO: 131 - DNA, 20 bp, Artificial
  synthetic pcr primer
atacaccacgttcccagtga

SEQ ID NO: 132 - DNA, 21 bp, Artificial
  synthetic pcr primer
cccatccatccaaatctttac

SEQ ID NO: 133 - DNA, 23 bp, Artificial
  synthetic pcr primer
cagggatcagatttttatttatc

SEQ ID NO: 134 - DNA, 22 bp, Artificial
  synthetic pcr primer
catgtcatacaaaagggcatga

SEQ ID NO: 135 - DNA, 22 bp, Artificial
  synthetic pcr primer
gaaatgttatgcctatgttgca

SEQ ID NO: 136 - DNA, 31 bp, Artificial
  synthetic pcr primer
aagcttttttttttttttttttttttttttv

SEQ ID NO: 137 - DNA, 20 bp, Artificial
  synthetic pcr primer
atggatgatgatatcgccgc

SEQ ID NO: 138 - DNA, 20 bp, Artificial
  synthetic pcr primer
ctagaagcatttgcggtgga

SEQ ID NO: 139 - DNA, 21 bp, Artificial
  synthetic pcr primer
gacatggaagccatcacagac

SEQ ID NO: 140 - DNA, 22 bp, Artificial
  synthetic pcr primer
cgaccgttcagctggatattac

SEQ ID NO: 141 - DNA, 20 bp, Artificial
  synthetic pcr primer
taatacgactcactataggg

SEQ ID NO: 142 - DNA, 19 bp, Artificial
  synthetic pcr primer
ggaaacagctatgaccatg

SEQ ID NO: 143 - DNA, 24 bp, Artificial
  synthetic pcr primer
ccagcacaatgaagatcaagatca

SEQ ID NO: 144 - DNA, 19 bp, Artificial
  synthetic pcr primer
gggccggactcgtcatact

SEQ ID NO: 145 - DNA, 22 bp, Artificial
  synthetic pcr primer
tcgctactaccgattggatggt

SEQ ID NO: 146 - DNA, 23 bp, Artificial
  synthetic pcr primer
cacctacggaaaccttgttacga

(19) United States
(12) Patent Application Publication
(10) Pub. No.: US 2005/0142580 A1
(43) Pub. Date: Jun. 30, 2005

(54) Methods and probes for diagnosing a gynaecological condition

(76) Inventors: Sun Kuie Tay, Singapore (SG); WeiPing Hu, Singapore (SG)

Correspondence Address: HOWSON AND HOWSON;ONE SPRING HOUSE CORPORATION CENTER, BOX 457, 321 NORRISTOWN ROAD, SPRING HOUSE, PA, 19477, US

(21) Appl. No.: 10/933,118

(22) Filed: Sep. 2, 2004

Related U.S. Application Data
(60) Provisional application No. 60/500,072, filed on Sep. 3, 2003.

(51) Int. Cl. C12Q 1/68; C07H 21/04

(52) U.S. Cl. 435/6; 536/23.2

(57) ABSTRACT

DNA sequences are identified that are useful in the diagnosis of gynaecological conditions such as endometriosis. Some of the sequences have a high identity with gene of known function such as Pim-2 oncogenes, IGFBP-5, ribosomal protein L41, propsaponin, fibulin-1, DLX5, 11β hydroxysteroid dehydrogenase type 2, SET, and RhoE. Methods for diagnosing or monitoring the progression of a gynaecological condition such as endometriosis may use primers directed to the DNA sequences identified herein.

### CROSS REFERENCE TO RELATED APPLICATIONS

This application claims the benefit of the priority of U.S. Provisional Patent Application No. 60/500,072, filed Sep. 3, 2003.

### BACKGROUND OF THE INVENTION

The present invention relates to DNA sequences useful in the diagnosis of gynaecological conditions such as endometriosis. More specifically, the present invention relates to probes and methods for the utilisation of the differential expression of certain genes in the diagnosis of endometriosis and related conditions.

Pelvic endometriosis is a prevalent disease in women of reproductive age. It has a propensity to run a chronic and recurrent course after treatment, leading to debilitating chronic pelvic pain and infertility. The etiology and pathogenesis of endometriosis is controversial. A long held belief postulates that endometrial cells from retrograde menstruation are the origin of the disease. The molecular and cellular events that lead to the implantation and growth of these ectopic endometrial cells and development of endometriosis in these women have yet to be determined. There appear to be some aberrant expressions of growth factors, angiogenic factors and adhesion molecules, and the deficiency of immune system have been postulated to play important roles in development and progression of endometriosis. Endometriosis is most probably a multifactorial/polygenic disorder involving dysregulation of multiple genes in the ectopic endometrial cells.

Current methods for diagnosing endometriosis have a number of drawbacks. Diagnosis by physical examination is invasive and may be painful since it is generally performed during early menses, when implants are likely to be largest and most tender. The physician palpates for a fixed, retroverted uterus, adnexal and uterine tenderness, pelvic masses or nodularity along the uterosacral ligaments. A rectovaginal examination is also required to identify uterosacral, cul-de-sac or septal nodules.

However, most women with endometriosis have normal pelvic findings, and laparoscopy is necessary for definitive diagnosis.

Pelvic ultrasonography, computed tomography and magnetic resonance imaging are occasionally used to identify individual lesions, but these modalities are not helpful in assessing the extent of endometriosis. Even with direct visualization, diagnosis of endometriosis can be difficult. Lesions appear in multiple guises that are at times difficult to interpret. This diagnostic challenge is compounded by the unreliable correlation between clinical manifestations and surgical findings. A patient who is asymptomatic or has very mild symptoms may have extensive disease, whereas an infertile patient may have very few implants.

Thus, there exists a need for a more reliable and less invasive method of diagnosing endometriosis. The present invention overcomes or alleviates a problem of the prior art by providing a genetic based method of diagnosis.

The discussion of documents, acts, materials, devices, articles and the like is included in this specification solely for the purpose of providing a context for the present invention. It is not suggested or represented that any or all of these matters formed part of the prior art or were common general knowledge in the field relevant to the present invention as it existed before the priority date of each claim of this application.

### SUMMARY OF THE INVENTION

The applicants have identified a number of DNA sequences that are over expressed or under expressed in cells of a patient having a gynaecological condition such as endometriosis, adenomyosis or endometrioma. Such DNA sequences are identified in the attached Sequence Listing, which forms a part of this application. Accordingly, in one aspect, the present invention provides a DNA molecule or genetic product thereof for monitoring the progression of, or diagnosing, or determining the severity of a gynaecological condition, the DNA molecule comprising a nucleotide sequence of one or more of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or equivalents or fragments thereof.

In another aspect, the present invention provides primers for monitoring the progression of, or diagnosing, or determining the severity of a gynaecological condition, the primers comprising a nucleotide sequence of one or more of SEQ ID NO: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 214, 125, 126, 127, 128, 129, 30, 131, 132, 133, 134, 135, or equivalents or fragments thereof.

In a further aspect, the present invention provides a DNA molecule or genetic product thereof for monitoring the progression of, or diagnosing, or determining the severity of a gynaecological condition, the DNA molecule comprising a nucleotide sequence of one or more of GenBank accession numbers NM_{—}001904, AF126110, NM_{—}000094, NM_{—}001028, NM_{—}007104, NM_{—}021104, X51346, BC006226, NM_{—}006791, NM_{—}013293, AK094591, NM_{—}001533, NM_{—}005839, NM_{—}003011, NM_{—}020306, NM_{—}004450, BC018111, AF317228, NM_{—}001798, NM_{—}005168, NM_{—}003224, NM_{—}005000, AY007096, NM_{—}000196, NM_{—}025233, X84075, BC004275, NM_{—}018269, AK001814, XM_{—}031397, NM_{—}014179, NM_{—}152350, NM_{—}009280, L27560, AK001278, NM_{—}030939, AK021534, BC014498, BC011980, BC010281, AK026200, AK091133, or equivalents or fragments thereof.

Some of these sequences have a high identity with genes with known functions such as Pim-2 oncogenes, IGFBP-5, ribosomal protein L41, propsaposin, fibulin-1, DLX5, 11β hydroxysteroid dehydrogenase type 2, SET, and RhoE.

In a further aspect, the present invention provides a method for monitoring the progression of, or diagnosing, or determining the severity of a gynaecological condition in a subject, the method comprising determining the expression level of a gene comprising a nucleotide sequence described herein in the subject, and comparing the expression level of the gene to the expression level of the same or a similar gene obtained from a reference sample, wherein a positive diagnosis is made if the expression level in the gene is statistically different to that found in the reference sample.

In another aspect, the present invention provides a method for monitoring the progression of, or diagnosing, or determining the severity of a gynaecological condition, the method comprising the detection of a mutation in a gene, the mutation capable of producing a protein with a higher or lower biological activity than a protein from a non-mutated gene.

In yet a further aspect, the present invention provides a probe or primer for monitoring the progression of, or diagnosing, or determining the severity of a gynaecological condition that is capable of hybridising to a DNA molecule or genetic product thereof as described herein.

Also provided is a kit for monitoring the progression of, or diagnosing, or determining the severity of a gynaecological condition comprising a probe or primer as described herein.

Still a further aspect of the present invention provides a method of screening for a compound having efficacy in the treatment or prevention of a gynaecological condition, the method including determining whether the candidate compound is capable of normalizing the expression levels of a gene comprising a DNA molecule as described herein.

Another aspect of the present invention provides a method of screening for a compound having efficacy in the treatment or prevention of a gynaecological condition, said method including determining whether the candidate compound is capable of acting as an agonist or antagonist to the protein product of a gene comprising a DNA molecule as described herein.

Other aspects and advantages of the present invention are described further in the following detailed description of the preferred embodiments thereof.

### BRIEF DESCRIPTION OF THE DRAWINGS

FIG. 1 shows a schematic protocol for cDNA subtractive hybridisation. Subtractive hybridization was performed between 1^{st}-strand cDNA of driver and 2^{nd}-strand cDNA of tester.

FIG. 2 shows subtraction efficiency by depletion of human β-actin and enrichment of the doped gene CAT in the tester cDNA population. The tester cDNA and the eluted cDNA samples were amplified by PCR with β-actin and/or CAT-specific primers before and after subtractions. PCR products were run out on a 1% agarose gel. Cycle 0, 1, 2, and 3 represent the cDNA samples before subtraction and after 1^{st}, 2^{nd}, and 3^{rd} subtraction, respectively. The high level of β-actin cDNA in the eluted solution (lane 6) demonstrates that the subtractive effect, not degradation, reduced the β-actin cDNA level in the tester.

FIG. 3 shows screening procedures and numbers of cDNA clones screened and identified. a, the false positive clones or equally expressed cDNA clones; b, equally expressed or duplicate cDNA clones; c, duplicate clones or cDNA clones containing only human repeat sequence inserts, such as human Alu repeat sequence.

FIGS. 4A-4C show Northern blot and real-time PCR analysis of the cDNA clones selected from the subtractive cDNA libraries, confirming a high agreement between Northern blot hybridization and real-time PCR technique. FIG. 4A shows overexpressed candidate genes in endometriosis vs. the paired uterine endometrium confirmed by Northern hybridization. The lanes marked a contain 4 μg of total RNA samples from uterine endometrium. The lanes marked b contain 4 μg of total RNA samples from endometriosis. β-actin was used as control for normalization. FIG. 4B shows underexpressed candidate genes in endometriosis vs. the paired uterine endometrium confirmed by Northern hybridization. FIG. 4C shows linear regression analysis of gene expression data determined by real-time PCR and by Northern blot analysis using the same paired RNA samples, as shown in FIG. 4A and FIG. 4B.

FIGS. 5A-5D show analysis of differential expression data of 76 candidate genes in 15 paired tissue samples from human endometriosis and autologous uterine endometrium generated by real-time PCR. FIG. 5A is a scatter plot of expression data of 76 genes in 15 paired samples after log₂-transformation. The upper line represents that y=2x, and the lower line represents that y=0.5x. FIG. 5B is a TreeView cluster that shows hierarchical clustering of gene expression data of 76 candidate genes (rows) in 15 pairs of clinical samples (columns). Each square represents a ratio of gene expression level in endometriosis to that in uterine endometrium after log₂-transformation. Color intensity represent overexpression (middle color intensity)-, underexpression (lightest grey) and equal expression (black), respectively, of gene in endometriosis vs. the paired uterine endometrium. FIG. 5C is a zoom-in picture for the 14 best candidate genes selected by employing a combination of both the criteria of mean fold-change of 2.0 and P≦0.01 in 15 cases, showing the consistent patterns of their differential expression. FIG. 5D shows an analysis of 15 cases by re-clustering based on the expression data of three immediate-early genes EGR1, JUN and JUND.

### DETAILED DESCRIPTION OF THE INVENTION

Unless otherwise noted, technical terms are used according to conventional usage. Definitions of common terms in molecular biology may be found in Benjamin Lewin, Genes V published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). The nomenclature for DNA bases as set forth at 37 CFR.s.1.822 are used herein.

The applicants have identified a number of DNA sequences that are over expressed or under expressed in cells of a patient having a gynaecological condition such as endometriosis, adenomyosis or endometrioma. Accordingly, in one aspect the present invention provides a DNA molecule or genetic product thereof for monitoring the progression of, or diagnosing, or determining the severity of a gynaecological condition, the DNA molecule comprising a nucleotide sequence of one or more of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or equivalents or fragments thereof.

It has been shown that the expression of certain genes is altered in a patient suffering endometriosis. The identification of the DNA sequences described herein allows inter alia for the development of diagnostic probes, diagnostic methods, and screening assays to identify compounds useful in treating gynaecological disorders.

The skilled person understands that it is not necessary to use the exact sequences defined by SEQ ID NO: 1 to 45. If limited alterations are made to the DNA sequences described herein, the DNA molecules and genetic products thereof are still able to fulfil their functions in the context of the present invention. For example, it is known that many genes are present in alternate forms (i.e. alleles) in different individuals. A probe generated against one allele of a gene is very often able to detect a different allele. Accordingly, the present invention includes within its scope DNA molecules “equivalent” to those described in SEQ ID NO: 1 to 45.

Preferably, an equivalent molecule will be at least 90% identical to the sequences described herein. More preferably, an equivalent molecule will be at least 95% identical, and even more preferably it will be at least 97% identical to the sequences described herein.

Another indication that two nucleic acid molecules are equivalent is that the two molecules hybridize to each other under stringent conditions when one molecule is used as a hybridization probe, and the other is present in a biological sample. Specific hybridization means that the molecules hybridize substantially only to each other and not to other molecules that may be present in the genomic material. Stringent conditions are sequence dependent and are different under different environmental parameters. Generally, stringent conditions are selected to be about 5° C. to 2° C. lower than the thermal melting point (Tm) for the specific sequence at a defined ionic strength and pH. The Tm is the temperature (under defined ionic strength and pH) at which 50% of the target sequence hybridizes to a perfectly matched probe. In a preferred form of the invention an equivalent sequence will hybridise to a sequence described herein under stringent conditions.

The degeneracy of the genetic code also provides flexibility in the sequences that will be useful in the context of the present invention. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequence that all encode substantially the same protein.

The genetic product may be a RNA transcript, or a cDNA derived from the RNA transcript. The genetic product may be a protein. The ability to interchange amino acids in a protein without materially affecting structure or function also provides flexibility in biological systems. For example, substituting a hydrophobic amino acid residue for another hydrophobic amino acid residue will have little effect on the properties of the resultant protein.

As a result of the degeneracy of the genetic code and the ability for a protein to tolerate conservative substitutions, a gene coding for a protein in one person can be quite different to that in another. However, there will still be a certain percentage identity between allelic forms of a gene in a given species. It is for this reason that the sequences described herein as well as equivalent sequences are useful in the context of the present invention.

It is also understood that fragments of the sequences described herein will be useful in the context of the present invention. Clearly, it is not necessary to utilize the entire sequence to practice the present invention. To be useful as a probe a sequence may be as little as 10 nucleotides long.

In a preferred form of the invention the DNA molecule comprises a nucleotide sequence of one or more of SEQ ID NO: 2, 6, 8, 11, 14, 17, 20, 24, 27, 28, 31, 34, 43 or 44 or equivalents or fragments thereof. Applicants have found that genes containing one or more of these sequences displayed a particularly high level of disregulation in subjects suffering from endometriosis.

After identifying sequences SEQ ID NOs: 1 to 45 as relevant to the pathogenesis and diagnosis of gynaecological disorders, the applicants performed further sequence analysis to identify the genes from which SEQ ID NO:1 to 45 originate. In screening the GenBank database, applicants have shown that many of SEQ ID NOs. 1 to 45 demonstrate identity with known genes and sequences. The present invention therefore also includes sequences that comprise these genes showing identity to SEQ ID NO 1 to 45. The present invention therefore also provides a DNA molecule or genetic product thereof for monitoring the progression of, or diagnosing, or determining the severity of a gynaecological condition, the DNA molecule comprising a nucleotide sequence of one or more of GenBank accession numbers NM_{—}001904, AF126110, NM_{—}000094, NM_{—}001028, NM_{—}007104, NM_{—}021104, X51346, BC006226, NM_{—}006791, NM_{—}013293, AK094591, NM_{—}001533, NM_{—}005839, NM_{—}003011, NM_{—}020306, NM_{—}004450, BC018111, AF317228, NM_{—}001798, NM_{—}005168, NM_{—}003224, NM_{—}005000, AY007096, NM_{—}000196, NM_{—}025233, X84075, BC004275, NM_{—}018269, AK001814, XM_{—}031397, NM_{—}014179, NM_{—}152350, NM_{—}009280, L27560, AK001278, NM_{—}030939, AK021534, BC014498, BC011980, BC010281, AK026200, AK091133, or equivalents or fragments thereof.

For example, SEQ ID NO:17 shows identity with the Pim-2 oncogene, which encodes a serine/threonine kinase. It also shows a considerable identity to Pim-1 oncogene, another close-related member of the family. Pim-2 mRNA is highly expressed in various human cancer cells, suggesting the possible involvement of Pim-2 in the transformation processes. Pim-1 and Pim-2 have been identified as common proviral insertion sites in lymphomas induced by MuLV. Pim kinases exhibit strong synergy with c-myc in tumorigenesis, cell proliferation and anti-apoptosis. In human diseases, Pim-1 has recently been shown to be a prognostic biomarker and a co-transcriptional oncogenic factor to myc in prostate cancer. Without wishing to be limited by theory the applicant's finding of over-expression of Pim-2 in over 90% of endometriosis patients provides the molecular basis for the synergistic transforming effect to take place in the ectopic tissue, conferring upon such tissue survival and progression properties.

SEQ ID NO:34 shows identity with a mRNA sequence L27560 (GenBank accession number L27560), which was initially misnamed as IGFBP-5. This gene was closely linked to two members of the IGFBP family and was mapped to chromosome 2q35 between the loci of IGFBP-5 and IGFBP-2 genes. Actually, the 5′-end of the reference mRNA sequence is partially overlapped with the 3′-end (exon 4) of IGFBP-5 gene. Besides structural overlapping, IGFBP-5 also exhibited high over-expression in endometriosis, which follows the same pattern as EA30 gene in the 15 patient cases discussed in the examples below with a correlation coefficient of 0.86 (data not shown). Their expression regulation appears to be governed by a similar mechanism in endometriosis, indicating that EA30 may be functionally related to IGFBP-5 gene.

SEQ ID NO:6 has identity with the gene encoding a highly basic ribosomal protein L41, which has been identified as a cellular factor capable of interacting with the CK2β and regulating the CK2 activity. Protein Ser/Thr kinase CK2 is one of the key cellular signals for cell survival, growth and proliferation. CK2 participates in a complex series of cellular functions by modulating the stability and/or activity of various important cellular proteins, such as DNA topoisomerase II, p53, NF-κB, IκB, β-catenin and proapoptosis protein Bid. It has been demonstrated that CK2 exhibits the elevated expression in various cancers and is implicated in tumorigenesis. RPL41 protein has been shown to stimulate the phosphorylation of DNA topoisomerase IIα by CK2 and to enhance the autophosphorylation of CK2α. Without wishing to be limited by theory, applicants propose that over-expression of RPL41 leads to the enhanced activity of CK2 in endometriosis. The up-regulation of RPL41 plays an important role in pathogenesis of endometriosis via modulating the CK2 activity.

SEQ ID NO:27 shows identity with Prosaposin, the glycoprotein precursor of saposins A, B, C, and D, which activate lysosomal hydrolysis of sphingolipids. Prosaposin exists in various tissues and body fluids and is especially abundant in the nervous system. Genetic defects in prosaposin have been associated with human lysosomal sphingolipid storage disorders. A recent study on Globoid cell leukodystrophy in a saposin A^{-/-} mouse model demonstrated that pregnancy dramatically alleviated the clinical and pathological phenotype of the affected mice. The estrogen supplementation produced similar protective effects to pregnancy, indicating that estrogen may, to certain extent, complement the deficiency of saposin A in the mice. Prosaposin was found to be stimulated dose-dependently by estrogen and was secreted by several breast cancer cells. It has been shown that prosaposin mRNA was highly expressed in the adult and embryonic gonads of both male and female mice, suggesting an important role in the reproductive systems. In human breast and ovarian cancer cells, prosaposin interacted with procathepsin D intracellularly and extracellularly, suggesting an involvement in tumor invasiveness and metastasis. In addition, prosaposin treatment of pheochromocytoma cells (PC12) induced extracellular signal-regulated kinases (ERKs) activity, stimulated DNA synthesis, and prevented cell death. Without wishing to be limited by theory, applicants propose that the elevated expression of prosaposin in endometriosis contributes to the survival, proliferation and invasiveness of endometrial cells on the ectopic locations.

SEQ ID NO:2 shows identity with the Fibulin-1 protein which is a multifunctional extracellular matrix protein that strongly interacts with fibronectin and suppresses fibronectin-regulated cell adhesion and motility. Over-expression of fibulin-1 in endometriosis suggests the similar mechanism is involved in the pathogenesis of this benign and estrogen-dependent disease.

SEQ ID NO:8 shows identity with the DLX5 gene which encodes a homeodomain transcription factor. It is one of six known human DLX homeobox genes, which are required for neurogenesis, skeleton and appendage development, and bone formation. The functions of different members of the DLX family appear to be partially redundant and the targets of DLX regulation include the DLX genes themselves. It has recently been shown that a homeotic transformation of lower jaws to upper jaws occurred in the Dlx5/6−/− mutant mice. Accumulating evidences suggest that DLX5 plays a crucial role in bone development and formation by modulating the gene expression of osteocalcin, COL1A1 and bone sialoprotein. Over-expression of DLX5 in the chicken calvarial osteoblasts stimulated osteoblastic differentiation that does not normally happen in vitro. Ectopic expression of DLX5 in rat osteosarcoma cells also induced up-regulation of fibronectin, suggesting an involvement in cell adhesion. The marked down-regulation of DLX5 expression by up to 92 fold in all 15 endometriosis cases of the following examples indicates that dysregulated DLX5 gene is involved in this pathological process and withdrawal of the effect of DLX5 product facilitates persistence of endometriosis cells in the proliferative state on the ectopic locations by failure of entering end stage differentiation and/or by interference of normal cell adhesion.

SEQ ID NO:24 shows identity with the HSD11B2 gene which encodes 11β-hydroxysteroid dehydrogenase type 2 (11β-HSD2), which inactivates glucocorticoid by converting cortisol to cortisone. It plays an important role in modulating glucocorticoid action within a given tissue by pre-receptor regulation, in collaboration with 11β-hydroxysteroid dehydrogenase type 1 (11β-HSD1). It has been shown that glucocorticoid was able to stimulate expression of P450 aromatase, which converts C19 steroids to estrogens, in human ovarian surface epithelial cell, leiomyoma and adipose tissue. Taken together, these findings indicate that the dysregulated expression of 11β-HSD2 plays a role in the pathogenesis of endometriosis via pre-receptor modulation of glucocorticoid action within the ectopic tissue.

SEQ ID NO:14 shows identity with the SET gene which encodes the protein I₂^{PP2A}, a potent and specific inhibitor of protein phosphatase 2A (PP2A). PP2A is a major protein Ser/Thr phosphatase expressed in all eukaryotic cells. It is involved in diverse cellular processes including cytokine signaling, transcription and translation, and is an important regulator of cell growth and apoptosis. It has been shown that the activity of phosphoprotein phosphatases including PP2A was required for the cAMP-induced expression of steroidogenic acute regulatory (StAR) protein and steroidogenesis. PP2A and SET have also been identified as posttranslational regulators of androgen biosynthesis by Cytochrome P450c17. In addition, PP2A was a binding partner of estrogen receptor α (ERα) and modulated the estrogen-independent activation of ERα by kinase-mediated phosphorylation. More interestingly, it has been demonstrated that Pim kinases were regulated by PP2A at the posttranslational level and inhibition of PP2A activity by okadaic acid stabilized the Pim proteins. Without wishing to be limited by theory, applicants propose that by modulating the PP2A activity, down-regulated SET expression positively contributes to the steroidogenesis and modulates the action of steroid hormones in the endometriotic lesion, whereas it partially alleviates the transforming effect of up-regulation of Pim-2 mRNA (the first candidate gene discussed above) via affecting the protein stability in the pathogenesis of endometriosis.

SEQ ID NO:20 shows identity with the RhoE gene encodes RhoE/Rnd3 protein, a member of the Rho family of Ras-related GTPases, which regulate the organization of the actin cytoskeleton in response to extracellular growth factors. RhoE binds GTP, but has no intrinsic GTPase activity and is found constitutively in the activated GTP-bound form. Expression of RhoE in mammalian cells inhibits the formation of actin stress fibers, membrane ruffles, and integrin-based focal adhesions, and induces loss of cell-substrate adhesion leading to cell rounding. RhoE may act to inhibit signaling downstream of RhoA and possess a RhoA antagonistic cell function. It has been shown that RhoA protein is over-expressed in breast cancers as compared to the paired normal tissues and the protein level correlates with tumor progression. Without wishing to be limited by theory, applicants propose that down-regulated RhoE contributes to the pathogenesis of endometriosis by altering cell adhesions and/or by mimicking the effect of enhanced RhoA protein expression in breast cancers.

The applicants have defined not only cDNA sequences, but also proteins, and classes of proteins that are involved in the pathogenesis of endometriosis. The present invention therefore includes the use of any sequence encoding a protein that exhibits identity with any one of SEQ ID NO: 1 to 45. Similarities between SEQ ID NO: 1 to 45 with other sequences have been found, and are detailed in Table 1 below.

TABLE 1
Characteristics and chromosome mapping of cDNA clones
identified from subtractive cDNA libraries

| | | | | | | |
|---|---|---|---|---|---|---|
| cDNA | Size | Accession | Map | Sequence | | Accession |
| clone | (bp) | number | locus | homology | Description | number |

Extracellular matrix/cell adhesion proteins

| | | | | | | |
|---|---|---|---|---|---|---|
| EA01 | 463 | BU197985 | 3p21 | CTNNB1 | β-catenin (cadherin-associated | NM_001904 |
| | | | | | protein) | |
| EA05 | 396 | BU197989 | 17q21 | COL1A1 | Collagen, type I, alpha 1 | NM_000088 |
| EA29 | 313 | BU198013 | 21q13 | FBLN1 | Fibulin-1 isoform D precursor | AF126110 |
| EA52 | 225 | BU198036 | 3p21.1 | COL7A1 | Collagen, type VII, alpha 1 | NM_000094 |
| EA77 | 344 | BU198061 | 17q21 | COL1A1 | Collagen, type I, alpha 1 | NM_000088 |

Ribosomal proteins

| | | | | | | |
|---|---|---|---|---|---|---|
| EA07 | 507 | BU197991 | 8q13.2 | RPL7 | Ribosomal protein L7 | NM_000971 |
| EA09 | 506 | BU197993 | 15q24 | RPS17 | Ribosomal protein S17 | NM_001021 |
| EA10 | 500 | BU197994 | 4p13 | RPL9 | Ribosomal protein L9 | NM_000661 |
| EA12 | 587 | BU197996 | Xq13.1 | RPS4X | Ribosomal protein S4, X-linked | NM_001007 |
| EA15 | 511 | BU197999 | 11q25 | RPS25 | Ribosomal protein S25 | NM_001028 |
| EA16 | 501 | BU198000 | 6p21.3 | RPL10A | Ribosomal protein L10a | NM_007104 |
| EA19 | 331 | BU198003 | 12q13 | RPL41 | Ribosomal protein L41 | NM_021104 |
| EA61 | 630 | BU198045 | 19q13.3 | RPL13A | Ribosomal protein L13a (a cell | NM_012423 |
| | | | | | proliferation inhibitor) | |

Transcription regulators

| | | | | | | |
|---|---|---|---|---|---|---|
| EA22 | 320 | BU198006 | 19p13.1 | JUN-D | jun D proto-oncogene | X51346 |
| EA26 | 527 | BU198010 | 5q31.1 | EGR1 | Early growth response 1 | NM_001964 |
| EA33 | 333 | BU198017 | 7q21 | DLX5 | Distal-less homeo box 5 | BC006226 |
| EA35 | 438 | BU198019 | 4p16 | CTBP1 | C-terminal binding protein 1 | XM_042659 |
| EA40 | 153 | BU198024 | 1p32 | JUN | c-jun proto-oncogene | NM_002228 |
| EA63 | 440 | BU198047 | 15q24 | MRG15 | MORF-related gene 15 | NM_006791 |

RNA processing and pre-mRNA splicing factors

| | | | | | | |
|---|---|---|---|---|---|---|
| EA14 | 369 | BU197998 | 7p15 | HTR2A | Similar to Homo sapiens | NM_013293 |
| | | | | | transformer-2 α | |
| EA39 | 567 | BU198023 | 12q13 | FLJ13467 | Similar to hnRNP-E2 | AK023529 |
| EA53 | 234 | BU198037 | 19p13.3 | FLJ37272 | Highly similar to GRG PROTEIN | AK094591 |
| EA54 | 237 | BU198038 | 19q13.1 | HNRPL | Heterogeneous nuclear | NM_001533 |
| | | | | | ribonucleoprotein L | |
| EA59 | 302 | BU198043 | 1p36.11 | SRRM1 | Serine/arginine repetitive matrix 1 | NM_005839 |
| | | | | | (a coactivator of pre-mRNA | |
| | | | | | splicing) | |
| EA76 | 169 | BU198060 | 1p35 | HPRP8BP | U5 snRNP-specific 40 kDa protein | NM_004814 |

Signaling intermediates

| | | | | | | |
|---|---|---|---|---|---|---|
| EA24 | 531 | BU198008 | 17p13.3 | YWHAE | 14-3-3 protein, epsilon | NM_006761 |
| | | | | | polypeptide | |
| EA27 | 410 | BU198011 | 9q34 | SET | A heat-stable protein phosphatase | NM_003011 |
| | | | | | 2A-specific inhibitor (I2PP2A) | |
| EA28 | 292 | BU198012 | 22q13 | TOMM22 | Translocase of outer mitochondrial | NM_020243 |
| | | | | | membrane 22 homolog (yeast) | |
| EA31 | 175 | BU198015 | 8q22 | YWHAZ | 14-3-3 protein, zeta polypeptide | NM_003406 |
| EA36 | 220 | BU198020 | 2p25 | Adam17 | Rat disintegrin and | NM_020306 |
| | | | | | metalloproteinase domain 17 | |
| EA43 | 235 | BU198027 | 2p23 | FLJ13786 | Highly similar to Mus musculus | AK023848 |
| | | | | | mRNA for ubiquitin conjugating | |
| | | | | | enzyme | |
| EA57 | 223 | BU198041 | 14q24.1 | ERH | Enhancer of rudimentary homolog | NM_004450 |
| | | | | | (Drosophila) | |
| EA62 | 397 | BU198046 | Xp11.23 | PIM2 | Pim-2 oncogene | XM_010208 |

Cell cycle

| | | | | | | |
|---|---|---|---|---|---|---|
| EA37 | 319 | BU198021 | 9q22.1-q22.3 | SPIN | Spindlin 1 | AF317228 |
| EA50 | 190 | BU198034 | 12q13 | CDK2 | Cyclin-dependent kinase 2 | NM_001798 |

GDP/GTP binding proteins

| | | | | | | |
|---|---|---|---|---|---|---|
| EA55 | 316 | BU198039 | 14q21 | ARF6 | ADP-ribosylation factor 6 | BC008918 |
| EA58 | 261 | BU198042 | 2q23.3 | ARHE | Ras homolog gene family, | NM_005168 |
| | | | | | member E | |
| EA70 | 77 | BU198054 | 20q13.3 | ARFRP1 | ADP-ribosylation factor related | NM_003224 |
| | | | | | protein 1 (Ras-related GTPase) | |

Metabolism

| | | | | | | |
|---|---|---|---|---|---|---|
| EA06 | 401 | BU197990 | 7q31 | NDUFA5 | NADH dehydrogenase | NM_005000 |
| | | | | | (ubiquinone) 1 alpha subcomplex, | |
| | | | | | 5 (13 kD, B13) | |
| EA21 | 408 | BU198005 | 19q13.33 | LOC126133 | Similar to aldehyde | XM_058991 |
| | | | | | dehydrogenase 1 family, member | |
| | | | | | A2; retinaldehyde dehydrogenase 2 | |
| EA60 | 232 | BU198044 | 16q22 | HSD11B2 | 11-β hydroxysteroid | NM_000196 |
| | | | | | dehydrogenase 2 | |
| EA64 | 129 | BU198048 | 17q21 | NBP | Similar to nucleotide binding | NM_025233 |
| | | | | | protein | |

Other cellular functions

| | | | | | | |
|---|---|---|---|---|---|---|
| EA04 | 178 | BU197988 | 11p11.2 | MYBPC | Cardiac myosin binding protein-C | X84075 |
| EA23 | 159 | BU198007 | 9q22-q31 | SEMA4D | Semaphorin 4D | NM_006378 |
| EA34 | 361 | BU198018 | 1q23 | ATP1B1 | ATPase, Na+/K+ transporting, | NM_001677 |
| | | | | | beta 1 polypeptide | |
| EA41 | 127 | BU198025 | 10q22.1 | PSAP | Prosaposin | XM_045140 |
| EA44 | 264 | BU198028 | 2p25.3 | SIPL | A hepatic factor supporting | NM_018269 |
| | | | | | hepatitis C virus replication | |
| EA78 | 301 | BU198062 | 3p25 | IMAGE: | Similar to ARPC4 | BC012596 |
| | | | | 429689 | | |

Unknown function

| | | | | | | |
|---|---|---|---|---|---|---|
| EA02 | 605 | BU197986 | 10q21 | IMAGE: | Uncharacterized | BC018658 |
| | | | | 4082362 | | |
| EA03 | 330 | BU197987 | 10p15 | IMAGE: | Uncharacterized | BC015987 |
| | | | | 4096273 | | |
| EA11 | 391 | BU197995 | 2p22 | FLJ10952 | Uncharacterized | AK001814 |
| EA13 | 379 | BU197997 | 1p36.32 | KIAA0495 | Uncharacterized | XM_031397 |
| EA17 | 196 | BU198001 | 1p36.12 | HSPC157 | cDNA clone from CD34+ stem | NM_014179 |
| | | | | | cells | |
| EA18 | 473 | BU198002 | 17p11.2 | TSAP19 | mRNA activated in tumor | AJ012499 |
| | | | | | suppression | |
| EA25 | 493 | BU198009 | 18q11.2 | Ss18 | Similar to Mus musculus synovial | NM_009280 |
| | | | | | sarcoma translocation, | |
| | | | | | Chromosome 18 | |
| EA30 | 207 | BU198014 | 2q35 | LOC151361 | Uncharacterized | XM_098048 |
| EA32 | 290 | BU198016 | 20q13.33 | FLJ20602 | Hypothetical protein | AK000609 |
| EA38 | 195 | BU198022 | 1q23.3 | FLJ10416 | Uncharacterized | AK001278 |
| EA42 | 373 | BU198026 | 6p22 | FLJ12619 | Hypothetical protein | NM_030939 |
| EA45 | 229 | BU198029 | 3p14 | A430107N12 | Uncharacterized, from RIKEN | AK020781 |
| | | | | | Mus full-length enriched library | |
| EA46 | 223 | BU198030 | 7p22.2 | IMAGE: | Uncharacterized | BC027714 |
| | | | | 4128750 | | |
| EA49 | 294 | BU198033 | 2q33.1 | FLJ13448 | Hypothetical protein | BC022453 |
| EA51 | 246 | BU198035 | 12q14 | FLJ11472 | Uncharacterized | AK021534 |
| EA56 | 507 | BU198040 | 11q12 | IMAGE: | Uncharacterized | BC014498 |
| | | | | 4856273 | | |
| EA65 | 135 | BU198049 | 5q22 | IMAGE: | Uncharacterized | BC013250 |
| | | | | 3867502 | | |
| EA66 | 254 | BU198050 | 1p34.1 | SP192 | Hypothetical protein SP192 | NM_021639 |
| EA67 | 420 | BU198051 | 5q23 | FLJ21409 | Uncharacterized | AK025062 |
| EA68 | 293 | BU198052 | 5q32 | HLCDGP1 | Down-regulated in lung cancer | NM_018548 |
| EA69 | 217 | BU198053 | 12p13 | IMAGE: | Uncharacterized | BC011980 |
| | | | | 3860421 | | |
| EA71 | 204 | BU198055 | 16p12 | IMAGE: | Uncharacterized | BC010281 |
| | | | | 3048642 | | |
| EA72 | 374 | BU198056 | 2q34 | KIAA0981 | KIAA0981 protein | XM_028867 |
| EA74 | 257 | BU198058 | 4q21 | FLJ22547 | Uncharacterized | AK026200 |
| EA75 | 322 | BU198059 | 22q12 | FLJ33814 | Uncharacterized | AK091133 |

Novel genes (match human genomic sequence)

| | | | | | | |
|---|---|---|---|---|---|---|
| EA08 | 491 | BU197992 | 12q15 | BAC clone | Novel | AC121761 |
| EA20 | 367 | BU198004 | 2p21 | BAC clone | Novel | AC073082 |
| EA47 | 222 | BU198031 | 8q23 | BAC clone | Novel | AP000427 |
| EA48 | 288 | BU198032 | 5p13 | BAC clone | Novel | AC008768 |
| EA73 | 342 | BU198057 | 9q34 | BAC clone | Novel | AL354944 |

In another aspect the present invention provides a method for monitoring the progression of, or diagnosing, or determining the severity of a gynaecological condition in a subject, the method comprising determining the expression level of a gene comprising a nucleotide sequence of any of SEQ ID NOs: 1 to 45, or equivalents or fragments thereof in the subject, and comparing the expression level of the gene to the expression level of the same or a similar gene obtained from a reference sample, wherein a positive diagnosis is made if the expression level in the gene is statistically different to that found in the reference sample.

The method may be used to diagnose a new gynaecological condition in a subject, or provide further information on an existing gynaecological condition. In both cases the method can be used to provide prognostic information for use by the clinician to assist in the management of the condition.

The method could use any one or more of a number of biological samples from the subject as a source for determining the expression level of the gene. In a preferred embodiment of the method the biological sample is tissue biopsy material from the vagina, uterus, cervix, fallopian tube or ovary. The biological sample may also be a fluid such as blood, saliva, urine, or a secretion of the reproductive tract. Preferably the sample is a biopsy of uterine endometrial tissue.

Where determination of the expression level involves isolating or purifying a polynucleotide or protein from the biological sample, the skilled artisan will be able to select an appropriate method. As an example, suitable techniques are described by Maniatis, T., Fritsch, E., F. and Sambrook et al., MOLECULAR CLONING, A LABORATORY MANUAL, 2nd Ed. Cold Spring Harbor Laboratory Press, Cold Spring Harbor. N.Y. (1989)

The expression level of the gene may be determined by any method familiar to the skilled person. One way of determining the expression level of a gene is to quantitate the amount of mRNA that has been transcribed from the relevant gene. This can be achieved by Northern blotting techniques for example. The quantitation could be determined on an absolute scale, or a relative scale.

The method of diagnosis requires that a judgement as to whether the gene is under expressed, or over expressed by comparison to a reference value for the same or similar gene. The reference value could be obtained from another sample from the subject that would be unlikely to be involved in a gynaecological condition. In a preferred form of the invention the reference value is obtained from eutopic endometrium taken from the subject.

Alternatively, the reference value could be an expressing level or average of a number of expression levels determined using biological samples taken from one or more subjects not suffering a gynaecological condition.

In a preferred embodiment of the method a positive diagnosis is made if at least a two-fold difference is noted between the expression level of the test sample and the reference sample. Based on further experience with the diagnostic method the skilled person may arrive at other cut off values. However, determining more appropriate cut off values is well within the ability of the skilled person.

In a preferred embodiment of the invention the method considers the expression level of a gene that is over expressed by at least a factor of two (i.e. SEQ ID NO: 2, 6, 11, 17, 27, 28, 34, 43, or 44). In a further preferred embodiment, the method considers the expression of a gene that is under expressed by a factor of at least two (i.e. SEQ ID NO: 8, 24, 14, or 20). In a further preferred embodiment, the method considers the expression of a gene that is under expressed by a factor of 15 (i.e., SEQ ID NO: 8).

In a preferred form of the invention the expression level is determined by analysis of a gene transcript, such as Northern blotting, quantitative PCR or sequencing.

In a further preferred form of the invention the expression level is determined by analysis of a protein encoded by the gene. In a more highly preferred embodiment the analysis of the protein is by Western blot, ELISA, surface plasmon resonance, or amino acid sequencing.

The skilled person will understand that the expression of more than one gene is likely to provide a more definitive picture of the gynaecological condition. Accordingly, in a preferred method the expression levels of at least 5 genes are considered. In a more highly preferred method the expression levels of at least 10 genes are considered.

Another aspect of the present invention provides a method for monitoring the progression of, or diagnosing, or determining the severity of a gynaecological condition, the method comprising the detection of a mutation in a gene, the mutation capable of producing a protein with a higher or lower biological activity than a protein from a non-mutated gene.

It is possible that while a certain gene involved in a gynaecological condition is not under expressed or over expressed, the protein product has an amino acid sequence that affords it a higher or lower than normal biological activity. Thus, the expression level may appear normal, but a gynaecological condition will still result because of the abnormally high or low biological activity of the protein.

In another aspect the present invention provides a probe or primer for monitoring the progression of, or diagnosing, or determining the severity of a gynaecological condition that is capable of hybridising to a DNA molecule or genetic product thereof as described herein.

Nucleic acid probes and primers may readily be prepared based on the nucleic acids provided by this invention. A probe may comprise an isolated polynucleotide attached to a detectable label or reporter molecule. Typical labels include radioactive isotopes, ligands, chemiluminescent agents, and enzymes. Methods for labeling and guidance in the choice of labels appropriate for various purposes are discussed, e.g., in Sambrook et al. (1989).

Primers are short nucleic acids, preferably DNA oligonucleotides 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (1989). PCR primer pairs can be derived from a known sequence, for example, by using computer programs intended for that purpose such as Primer (Version 0.5, Whitehead Institute for Biomedical Research, Cambridge, Mass.). One of skill in the art will appreciate that the specificity of a particular probe or primer increases with its length. Thus, for example, a primer comprising 20 consecutive nucleotides of the gene of interest will anneal to a target sequence with a higher specificity than a corresponding primer of only 15 nucleotides. Thus, in order to obtain greater specificity, probes and primers may be selected that comprise 20, 25, 30, 35, 40, 50 or more consecutive nucleotides.

Given the DNA sequences provided in the instant specification, the skilled person is adequately enabled to produce a probe or primer useful in the methods described herein.

In a preferred form of the invention the primers comprise nucleotide sequences of one or more of SEQ ID NO: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 214, 125, 126, 127, 128, 129, 30, 131, 132, 133, 134, 135, or equivalents or fragments thereof.

Furthermore, a skilled person could deduce the protein that would be translated from the transcript, synthesise the protein (or fragment of the protein), and use it to generate antibody probes specific for that protein.

The present invention contemplates the use of one or a plurality of sequences. In a preferred embodiment of the invention a plurality of sequences are screened for abnormal expression in the patient. A clinician would be more convinced that a patient was in fact suffering from a gynaecological condition if abnormal expression levels were seen for a number of candidate genes.

In another aspect the present invention provides a kit for monitoring the progression of, or diagnosing, or determining the severity of a gynaecological condition comprising a probe or primer as described herein.

In another aspect the present invention provides a method of screening for a compound having efficacy in the treatment or prevention of a gynaecological condition, the method including determining whether the candidate compound is capable of normalizing the expression levels of a gene comprising a DNA molecule as described herein.

The present invention is not limited to the diagnosis or prognosis of gynaecological conditions, but will have use in the treatment of such conditions. The sequences identified by the applicants as important in pathogenesis point to a number of genes that are involved in the disease process. This allows further investigations into the identification of compounds that are able to modulate expression of the relevant genes allowing the expression levels to return to normal. For example, if a gene is over expressed in endometriosis, the aim would be to decrease expression. This could be accomplished by gene therapy methods (such as antisense technology). If a gene is under expressed, gene therapy could be used to augment production of the gene product.

Similarly, it would be possible to identify compounds that are capable of modulating the biological activity of a protein gene product encoded by a gene described herein. Another aspect of the present invention therefore provides a method of screening for a compound having efficacy in the treatment or prevention of a gynaecological condition, said method including determining whether the candidate compound is capable of acting as an agonist or antagonist to the protein product of a gene comprising a DNA molecule as described herein.

The invention will be applicable to many diseases and conditions of gynaecological origin that relate to the ectopic or uncontrolled growth of uterine tissue. Preferably, the uterine tissue is endometrium.

The gynaecological condition may be related to a disorder of any pelvic tissue such as uterine, fallopian, ovarian, cervical or vaginal tissue. It will be understood that tissues and other than the reproductive tissues may also be involved. For example, the ectopic growth of uterine tissue may occur in other organs or structures of the pelvis such as the ureters, bladder, urethra, kidney, peritoneum, mesentery, abdominal wall, diaphragm, stomach, liver, pancreas, intestine, rectum, blood vessel, or connective tissue. Conditions affecting these organs or structures are therefore included in the scope of the present invention.

In a preferred form of the invention the gynaecological condition to which the present invention applies includes, but is not limited to, endometriosis, adenomyosis and endometrioma.

The invention will now be further described in the following non-limiting examples.

### EXAMPLES

### Example 1

### Materials and Methods

Tissue specimens. Tissue biopsies of pelvic endometriosis and normal uterine endometrium were collected in pair from 15 patients undergoing laparoscopy or hysterectomy for endometriosis (Table 2). Portions of the tissue specimens were stored in liquid nitrogen immediately for this study while the remaining were used for histo-pathological diagnosis. The patients either had never received any hormonal treatment or had ceased any hormonal medication for at least 6 months before surgery. The patients were categorized by menstrual phases and severity of endometriosis. The proliferative (days 4-14) and secretory (days 16-28) phases of menstruation was determined by the patients' menstrual history and histo-pathological assessment of the endometrium. The severity or stage of evolution of endometriosis was based on surgical pathological findings according to the revised American Fertility Society classification system (Revised American Society for Reproductive Medicine classification of endometriosis: 1996. Fertil. Steril. 1997 May; 67(5):817-21). For analysis of gene expression profiles, the 15 pairs of samples were classified into two groups: (A) early disease (clinical stage I & II) and (B) advanced disease (clinical stage IlI & IV).

TABLE 2
Characteristics of clinical tissue biopsies used for gene
expression profiling by real-time PCR

| | Patient | | | |
|---|---|---|---|---|
| | No. | | | Stage of |
| Patient group | & symbol | Age (yr) | Cycle phase | endometriosis |
| Early disease | 1 (I-P1) | 33 | Proliferative | I |
| | 2 (I-P2) | 32 | Proliferative | I |
| | 3 (II-P1) | 28 | Proliferative | II |
| | 4 (II-P2) | 32 | Proliferative | II |
| | 5 (II-P3) | 34 | Proliferative | II |
| | 6 (II-S1) | 32 | Secretory | II |
| Advanced disease | 7 (III-P1) | 50 | Proliferative | III |
| | 8 (III-P2) | 43 | Proliferative | III |
| | 9 (III-P3) | 35 | Proliferative | III |
| | 10 (III-N) | 29 | Not available | III |
| | 11 (IV-P1) | 46 | Proliferative | IV |
| | 12 (IV-P2) | 41 | Proliferative | IV |
| | 13 (IV-S1) | 34 | Secretory | IV |
| | 14 (IV-S2) | 39 | Secretory | IV |
| | 15 (IV-S3) | 36 | Secretory | IV |

RNA preparation. The fresh-frozen tissue was weighted, rinsed with ice-cold PBS solution and minced on ice before RNA purification. Total RNA was prepared from 7-50 mg fresh-frozen tissue specimens using Triazole™ Reagent according to the protocol recommended by the manufacturer (Gibco BRL).

1^{st}-strand and 2^{nd}-strand cDNA synthesis. mRNA was purified from 30-40 μg of total RNA using the Dynabeads® Oligo (dT)₂₅ (DYNAL) reagent and 1^{st}-strand cDNA was synthesized (42° C., 50 min; 50° C., 30 min) with SuperScript II™ Reverse Transcriptase (Gibco BRL, 400 units) reagent using Dynabeads® Oligo (dT)₂₅ reagent as RT primer according to the protocol recommended by the manufacturer (DYNAL). All the reactions involving Dynabeads® reagent were performed on a roller mixer. After cDNA synthesis, the unprimed Oligo (dT)₂₅ on the Dynabeads® reagent were removed by T4 DNA polymerase (New England Biolabs, 3 units, room temperature, 1 hour) (9). 1^{st}-strand cDNA on Dynabeads® reagent was tailed with poly(dA)ₙ by Terminal Deoxynucleotidyl Transferase (Gibco BRL, 15 units, room temperature, 1 hour). A 2^{nd}-strand cDNA was then synthesized with Taq DNA polymerase (Qiagen) using a one-base anchor oligonucleotide HT₂₆V (AAGCTTTTTTTTTTTTTTTTTTTTTTTTTTV, V: A, G or C; SEQ ID NO: 136) as primer (94° C., 3 min; 55° C., 2 min and 68° C., 15 min). The 2^{nd}-strand cDNA was then eluted in 30 μl TE buffer with 0.6 μg carrier tRNA (Gibco BRL) from the 1^{st}-strand cDNA Dynabeads at 95° C., 2 min. The 1^{st}-strand cDNA Dynabeads® reagent was then removed with a magnet (Dynal MPC-E-1).

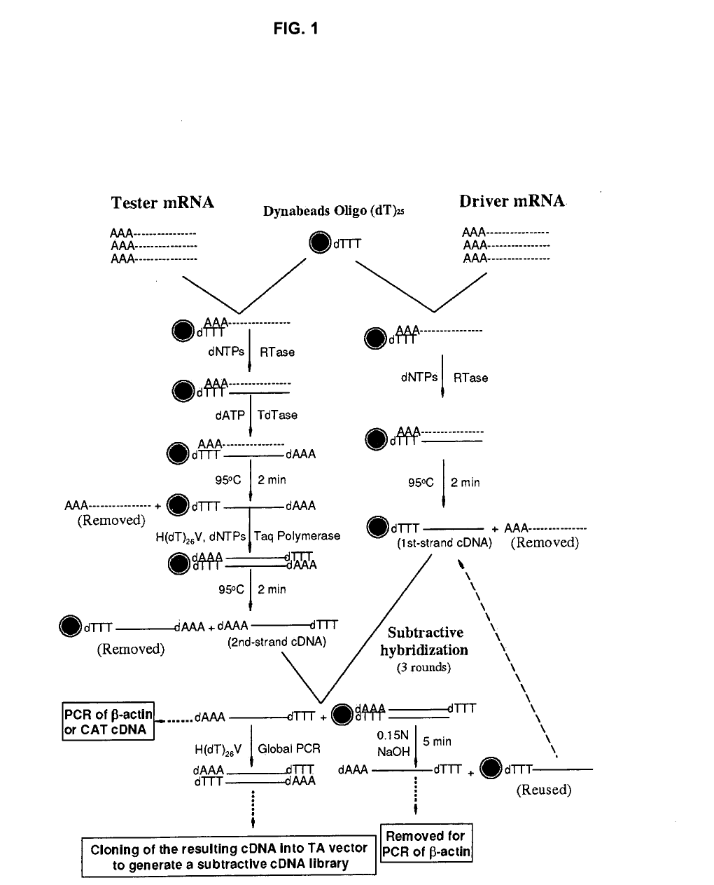

Subtractive cDNA library construction. Subtractive hybridization was performed in a small volume (≦30 μl) based on the solid-phase cDNA using a similar protocol as described by Lönneborg et al. 1995 PCR Methods Appl., 4(4):S168-176, with several modifications. FIG. 1 shows the schematic protocol for cDNA preparation and subtractive library construction used in this study. Briefly, the main modifications include: (a) 2^{nd}-strand cDNA was used as tester instead of mRNA; (b) subtractive progress was monitored by PCR amplification of β-actin gene in tester solution after each cycle of subtraction. Efficacy of the protocol was evaluated by duplex PCR of β-actin and CAT genes, representing common and differential sequences, respectively, in the differential cDNA model system, which consisted of a driver—1^{st}-strand cDNA Dynabeads® reagent and a tester—2^{nd}-strand cDNA from the same RNA doped with an extra CAT cDNA. PCR primers used for β-actin and CAT genes were as follows: (a) β-actin forward primer, 5′-ATGGATGATGATATCGCCGC-3′ SEQ ID NO: 137; (b) β-actin reverse primer, 5′-CTAGAAGCATTTGCGGTGGA-3′ SEQ ID NO: 138; (c) CAT forward primer, 5′-GACATGGAAGCCATCACAGAC-3′ SEQ ID NO: 139; and (d) CAT reverse primer, 5′-CGACCGTTCAGCTGGATATTAC-3′ SEQ ID NO: 140.

After subtractive hybridization using cDNA samples prepared from endometriosis and paired uterine endometrium, the remaining 2^{nd}-strand tester cDNA with a poly(dA)ₙ-tail in the hybridization solution was isolated by using Dynabeads® Oligo (dT)₂₅ reagent. The subtracted cDNA molecules were globally amplified by PCR using HT₂₆V universal primer. The PCR product was purified with the GFX™ PCR DNA and Gel Band Purification Kit (Amersham Pharmacia Biotech). The purified PCR product was then cloned into a pCR-XL-TOPO TA vector (Invitrogen), and transformed into TOP10 competent cells to generate a subtractive cDNA library. [0081] Screening of subtractive cDNA libraries by differential hybridization. 738 colonies were random-selected from the subtractive libraries for differential screening with colony and/or dot blot hybridization. About 400 colonies were prescreened by colony hybridization prior to dot blot hybridization. The rest colonies were directly screened by PCR and dot blot hybridization. For dot blot hybridization, cDNA inserts were amplified by colony-PCR using the vector-specific primers (T7 primer: 5′-TAATACGACTCACTATAGGG-3′ SEQ ID NO: 141; M13 Reverse primer: 5′-GGAAACAGCTATGACCATG-3′ SEQ ID NO: 142). PCR product was mixed with equal volume of 0.6 N NaOH to denature dsDNA. 2 μl of denatured DNA sample was transferred to Hybond-N+™ nylon membrane (Amersham Pharmacia Biotech) with a micropipettor. A 1128-bp human β-actin cDNA (coding region) and a vector without insert were used as the positive and negative controls, respectively. The blotted membranes were then neutralized with 0.5 M Tris-HCl (pH 7.5) and cross-linked using an UV Stratalinkert instrument (Stratagene). The cDNA probes prepared from paired endometriosis and endometrium samples were labeled with Digoxigenin-11-dUTP (Boehringer Mannheim). Colony and dot blot hybridization with the DIG-labeled probes and their chemiluminescent detection were performed according to the protocol recommended by the manufacturer (Boehringer Mannheim).

DNA sequencing. DNA sequencing was performed with ABI Prism BigDye™ Terminator Cycle Sequencing Ready Reaction Kit and ABI Prism® 310 Genetic Analyzer (PE Applied Biosystems). Cycle sequencing reaction was carried out with GeneAmp® PCR System 9700 (PE Applied Biosystems) using T7 and/or M13 Reverse sequencing primers.

Northern analysis. Total RNA samples (4-8 μg) were electrophoresed on 1.2% formaldehyde denaturing agarose gel prior to overnight capillary transfer to BM nylon membranes (positively charged, Boehringer Mannheim). The previously isolated cDNA clones were purified with Wizard® plus SV Minipreps DNA Purification System (Promega). DIG-DNA probes were then prepared from the purified cDNA clones by PCR using the vector-specific primers. The RNA blots were hybridized with DIG-DNA probes in DIG Easy Hyb solution (Boehringer Mannheim) at 50° C. overnight, followed by washes of 2×5 min in 2×SSC, 0.1% SDS at room temperature and 2×15 min in 0.1×SSC, 0.1% SDS at 68° C. under constant agitation. Chemiluminescent detection of the hybridized blots was performed according to the protocol recommended by the manufacturer (Boehringer Mannheim). Northern blots were quantified with densitometry and the expression data for target genes were then normalized to β-actin mRNA levels for further analysis. The extent of differential expression of these genes was determined by the fold-change of expression levels in endometriosis versus the paired uterine endometrium after normalization.

Gene expression profiling by real-time PCR. 4 μg of total RNA from each samples were treated with RNase-free DNase I (Gibco BRL) to eliminate the contaminant genomic DNA. The mixtures were directly used for reverse transcription with random hexamer primers and SuperScript First-Strand Synthesis System (Gibco BRL). Real-time PCR was performed with SYBR® Green Master Mix on an ABI Prism® 7700 Sequence Detection System according to the manufacturer's protocol (PE Applied Biosystems). The cDNAs were amplified by incubation for 10 minutes at 95° C. to activate the Hot Start AmpliTaq Gold® DNA polymerase reagent, followed by 45 cycles of denaturation at 94° C. for 30 seconds, annealing at 56° C. for 1 minutes and extension at 68° C. for 1 minutes. 15 ng of cDNA (total RNA equivalent) was used for each PCR reaction performed in 20 Eli with Optical 96-Well Reaction Plates (PE Applied Biosystems). Melting curves were generated after amplification to check the PCR specificity. Amplicon size and reaction specificity were further confirmed by electrophoresis on a 1.5% agarose gel and a single PCR product with expected size should be observed. The changes in fluorescence of the SYBRO® Green I dye in each cycle were monitored by ABI Prism® 7700 system, and the threshold cycle (C_{T}), which is defined as the cycle number at which the amount of amplified target reaches a fixed threshold, was obtained for each gene in each sample. Both primer sets for β-actin and 18S rRNA were included in each plate of PCR reactions as endogenous control and β-actin was used to normalize the quantity of RNA used. The C_{T} value of β-actin was subtracted from that of each target gene to obtain a ΔC_{T} value. The relative mRNA level of each target gene in each pair of samples was determined by 2^{-ΔΔCT}, where
ΔΔC_{T}=ΔC_{T}[Gene(x)_{endometriosis}]−ΔC_{T}[Gene(x)_{endometrium}],
ΔC_{T}[Gene(x)_{endometriosis]}=C_{T}[Gene(x)_{endomethosis}]−C_{T}[(β-actin)_{endometriosis}],
and ΔC_{T}[Gene(x)_{endometrium}]=C_{T}[Gene(x)_{endometrium}]−C_{T}[(β-actin)_{endometrium}].

The C_{T} values of each gene in endometriosis and the paired endometrium samples were determined in triplicate experiments and the mean C_{T} values were used for analysis. The real-time PCR reactions were repeated if the coefficient of variation (CV) for any C_{T} values was more than 4%.

Primers used for real-time PCR. Primers for target genes were designed based on the sequences of the cDNA clones and synthesized by OPERON (USA). The nucleotide sequences for 78 selected cDNA clones (EA01-EA78) and 76 pairs of primers (corresponding to clone EA01-EA76) have been submitted to the GenBank (GenBank accession number: BU197985-BU198062) and will be available at the Web site: http://www.ncbi.nlm.nih.gov. The information provided therein is incorporated herein by reference. The primer sequences for β-actin mRNA and 18S rRNA are as follows: (a) β-actin forward primer: 5′-CCAGCACAATGAAGATCAAGATCA-3′ SEQ ID NO: 143; (b) β-actin reverse primer: 5′-GGGCCGGACTCGTCATACT-3′ SEQ ID NO: 144; (c) 18S rRNA forward primer: 5′-TCGCTACTACCGATTGGATGGT-3′ SEQ ID NO: 145; and (d) 18S rRNA reverse primer: 5′-CACCTACGGAAACCTTGTTACGA-3′ SEQ ID NO: 146.

Data analysis. The differential gene expression measured by the relative quantitative values (fold changes) given by 2^{-ΔΔCT} for each target gene in each pair of samples were analyzed and displayed by hierarchical clustering algorithm after logarithmic (log₂) transformation using the Cluster and TreeView software (Eisen et al, 1998 Proc. Natl. Acad. Sci. USA, 95:147863-14868). The genes and clinical samples were grouped on the basis of similarities of gene expression patterns. Statistical analysis for difference of gene expression data between normal endometrium and endometriotic tissue samples was performed with paired Student's t test. Analyses with a P value of 0.05 or less were considered to be statistically significant.

Chromosome mapping. The candidate genes of interest were mapped to chromosomes by searching human genome, LocusLink and UniGene databases at the Web site: http://www.ncbi.nlm.nih.gov.

### Example 2

### Subtractive Hybridization, Library Construction and Screening with RNA Samples from Paired Endometrium and Endometriosis Tissues

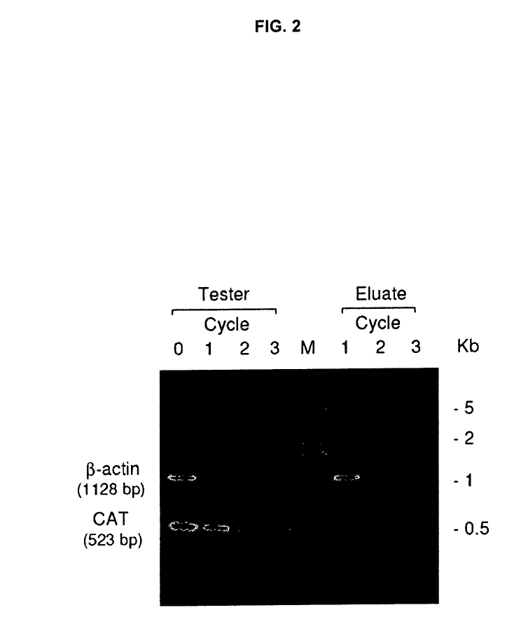

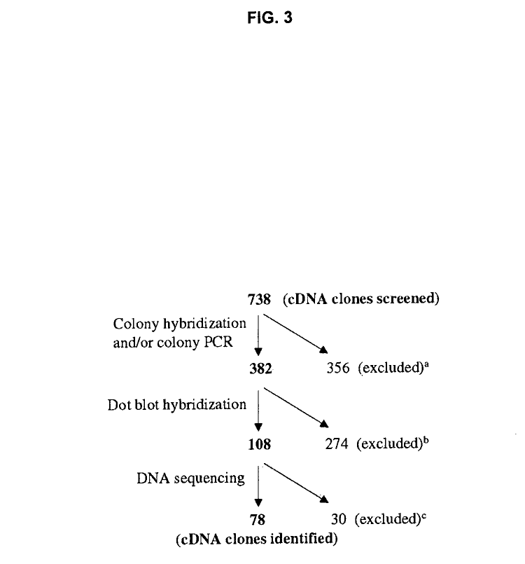

The efficacy of the subtractive protocol was confirmed experimentally (FIG. 2). The subtractive hybridization in two directions to enrich the genes over-expressed or underexpressed in endometriosis tissue generated a large number of cDNA clones. 738 colonies were screened by colony PCR and/or differential hybridization to remove those false positive clones, and duplicate or equally expressed genes. 108 cDNA clones were identified for further study with DNA sequencing (FIG. 3).

### Example 3

### DNA Sequencing Analysis, Gene Identities and Chromosome Mapping

DNA sequencing analysis through the GenBank databases for 108 cDNA clones isolated from the libraries revealed that 78 contained different cDNA inserts while the other 30 were duplicate clones or had the same inserts with different orientation and were therefore excluded. Of 78 cDNA clones, 48 were identical with or very similar to known genes in the GenBank databases, and 25 were matched to unknown genes, including uncharacterized human or mouse mRNA and hypothetical proteins. The remaining 5 clones matched human genomic sequences with no similarity with any known cDNA or mRNA, i.e. novel genes. The 48 known genes included 5 genes for extracellular matrix/cell adhesion proteins, 8 for ribosomal proteins, 6 for transcription regulators, 6 for RNA processing and pre-RNA splicing factors, 8 for signaling intermediates, 2 for cell cycle, 3 for GDP/GTP binding proteins, 4 for metabolism and 6 for other cellular functions. All these 78 genes were mapped to chromosomes by searching human genome, LocusLink and UniGene databases (see Table 1 above).

### Example 4

### Northern Analysis

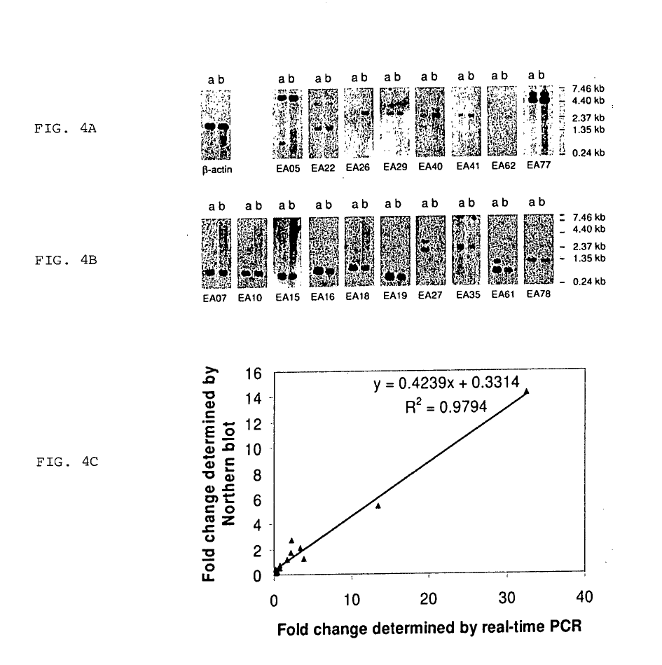

To verify the differential expression of the genes, those cDNA clones generating strong signals during differential screening were analyzed with Northern blot hybridization on the same pair of original RNA samples used for subtractive hybridization. This confirmed the differential expression of 18 genes in endometriosis versus the paired uterine endometrium (FIGS. 4A-4C). Of these 18 genes, 8 were over-expressed (FIG. 4A) and 10 were under-expressed (FIG. 4B) in the endometriotic sample. Notably, there was a marked over-expression in EA26 (EGR1), EA40 (JUN) and EA62 (PIM2) genes and under-expression in EA18 (TSAP19), EA27 (SET), EA35 (CTBP1) and EA61 (RPL13A) genes. Northern blots were quantified with densitometry and the expression data for target genes were then normalized to β-actin mRNA levels (FIG. 4A) for further analysis.

### Example 5

### Gene Expression Profiling by Real-time PCR

The CYBR Green-based real-time PCR technique was used for gene expression profiling in multiple clinical samples from patients (Table 2) with endometriosis at different stages (I-IV) after an evaluation study confirming a high agreement between real-time PCR and Northern blot hybridization (r²=0.986) (FIG. 3 at (c)). Some 8,000 real-time PCR reactions were performed to analyze the expression profiles of 76 out of 78 candidate genes in 15 pairs of clinical tissue samples.

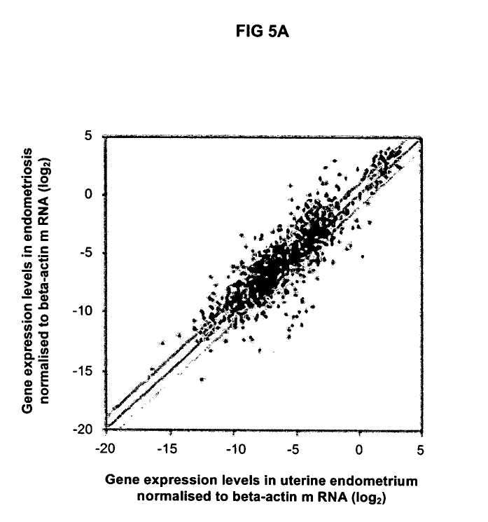

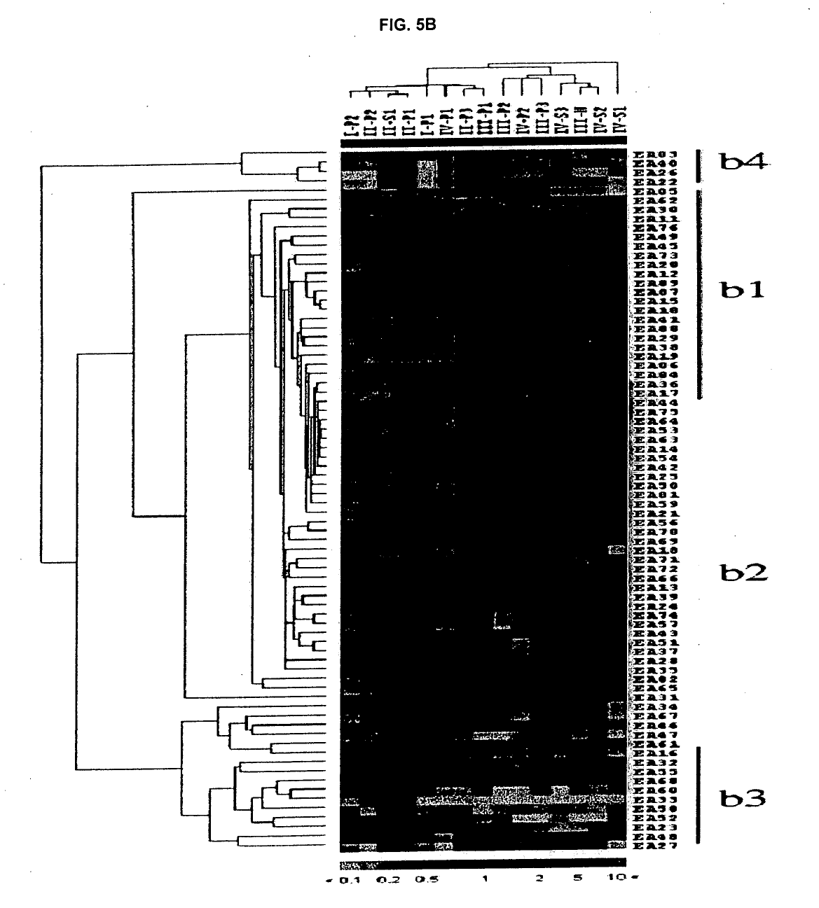

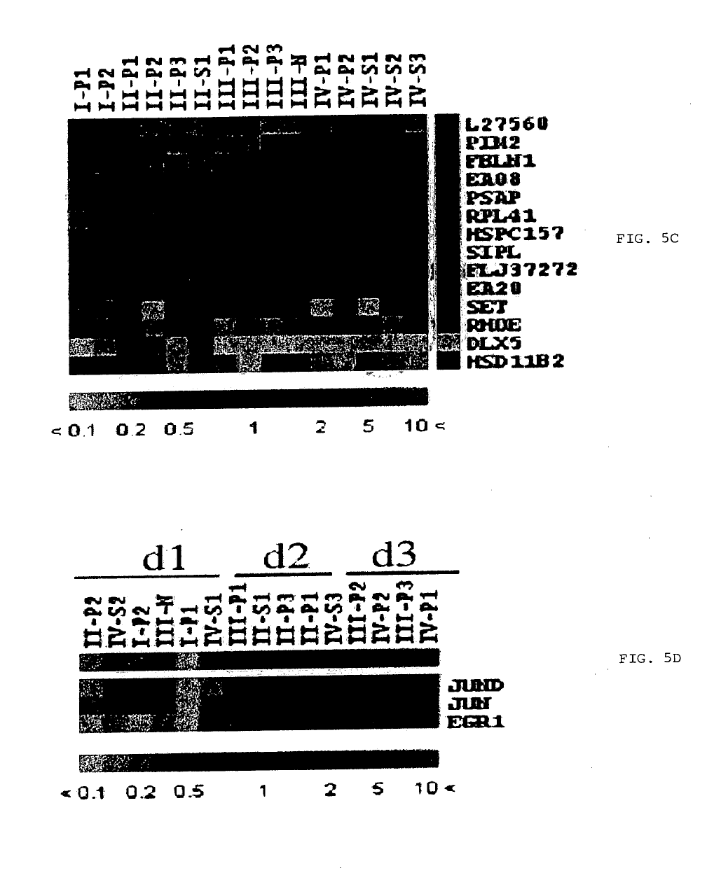

To obtain a general idea of differential regulation of 76 genes in 15 paired samples gene expression data generated by real-time PCR were displayed on a scatter plot (FIG. 5A). Of 1140 data points, 649 lay between the red and green lines and therefore their expression ratios in endometriosis samples vs. the paired uterine endometrium are within the range from 0.5 to 2.0. The other 491, representing 43% of the total data points, lying outside this range indicate that the genes were differentially expressed by over 2 fold in these paired clinical samples.

Two methods were used to identify differentially regulated genes:

(1) Using an arbitrary cutoff criteria. Of the 76 candidate genes, 30 (39%) showed consistent differential expression in more than 70% of cases (at least 11 out of 15). In fact, consistent differential expression was seen in 15 (100%) cases for 2 genes, in 14 (93%) cases for 3 genes, in 13 (87%) cases for 8 genes, in 12 (80%) cases for 9 genes, and in 11 (73%) cases for another 8 genes. Raising the cutoff threshold to 2-fold difference between endometriosis and autologous endometrium tissues, 14 (18%) of 76 genes were consistently differentially expressed in at least 9 (60%) of 15 cases.

(2) Using significant statistical difference as criteria. A quantitative assessment of the differential gene expression in terms of a relative expression ratio allowed the paired t-test to be used to test for statistically significant differential expression between endometriosis and the autologous endometrium tissues for 76 candidate genes (see Table 1 above). Employing a combination of both the criteria of mean fold-change of 2.0 and statistically significant ΔΔC_{T} with P≦0.01 in the 15 cases (Table 2), 14 best candidate genes were identified, including 10 genes over-expressed by 2.0 to 5.6 folds and 4 genes under-expressed by 2.2 to 15.2 folds in endometriosis. 12 of these 14 best candidates concurred with 12 of the 14 genes identified by using the arbitrary 2-fold cutoff method. These 14 candidates were therefore chosen for further investigation.

### Example 6

### Identification of Preferred Genes

The more detailed information including gene identity and chromosome mapping on the 14 best candidates was listed in Table 3. Briefly, 10 over-expressed genes include Pim-2 oncogene (Xp11.23), Ribosomal protein L41 (12q13), Prosaposin (10q22.1), Fibulin 1 (21q13), SIPL protein (2p25.3), three uncharacterized mRNAs: L27560 (2q35), HSPC157 protein (1p36.12) and FLJ37272 (19p13.3), and two novel genes EA08 (12q15) and EA20 (2p21). 4 under-expressed genes include Distal-less homeobox 5 protein (7q21), 11 beta-hydroxysteroid dehydrogenase type 2 (16q22), protein phosphatase 2A-specific inhibitor (SET) (9q34), and ras homolog gene family, member E (RhoE) (2q23.3).

TABLE 3
Summary of characteristics of the 14 best candidate genes
consistently differentially regulated in 15 paired samples

| | | | | | | |
|---|---|---|---|---|---|---|
| | Gene symbol | | Mean | | | |
| cDNA | (accession | | fold- | P | Map | Chromosomal |
| clone | No.) | Gene description | change | value | locus | aberrations* |

Overexpression in endometriosis

| | | | | | | |
|---|---|---|---|---|---|---|
| EA30 | LOC151361 | Uncharacterized | 5.6 | 0.0003 | 2q35 | Gain of Chr.2 |
| | XM_098048 | | | | | (CGH, 18) |
| EA62 | PIM2 | Pim-2 oncogene | 3.4 | 0.0002 | Xp11.23 | |
| | XM_010208 | | | | | |
| EA19 | RPL41 | Ribosomal protein | 3.2 | 0.0017 | 12q13 | |
| | NM_021104 | L41 | | | | |
| EA41 | PSAP | Prosaposin | 3.1 | 0.0000 | 10q22.1 | Trisomy 10 |
| | XM_045140 | | | | | (R-banding, |
| | | | | | | 20) |
| EA29 | FBLN1 | Fibulin 1 isoform | 3.0 | 0.0003 | 21q13 | Gain of 21q |
| | AF126110 | D precursor | | | | (CGH, 18) |
| EA08 | BAC clone | Novel gene | 2.7 | 0.0000 | 12q15 | |
| | AC121761 | | | | | |
| EA17 | HSPC157 | Uncharacterized | 2.4 | 0.0018 | 1p36.12 | |
| | NM_014179 | cDNA clone (from | | | | |
| | | CD34+ stem cells) | | | | |
| EA20 | BAC clone | Novel gene | 2.2 | 0.0038 | 2p21 | Gain of Chr.2 |
| | AC073082 | | | | | (CGH, 18) |
| EA53 | FLJ37272 | Uncharacterized | 2.2 | 0.0055 | 19p13.3 | Tetrasomy 19 |
| | AK094591 | (highly similar to | | | | (R-banding, |
| | | GRG PROTEIN) | | | | 20) |
| EA44 | SIPL | SIPL protein (a | 2.0 | 0.0041 | 2p25.3 | Gain of Chr.2 |
| | NM_018269 | hepatic factor | | | | (CGH, 18) |
| | | supporting | | | | |
| | | hepatitis C virus | | | | |
| | | replication) | | | | |

Underexpression in endometriosis

| | | | | | | |
|---|---|---|---|---|---|---|
| EA33 | DLX5 | Distal-less | 15.2 | 0.0000 | 7q21 | |
| | BC006226 | homeobox 5 | | | | |
| | | protein | | | | |
| EA60 | HSD11B2 | 11 beta- | 3.2 | 0.0023 | 16q22 | Loss of 16q |
| | NM_000196 | hydroxysteroid | | | | (CGH, 17); |
| | | dehydrogenase | | | | Monosomy 16 |
| | | type 2 | | | | (FISH, 19) |
| EA27 | SET | A heat-stable | 2.9 | 0.0015 | 9q34 | Loss of 9q |
| | NM_003011 | protein | | | | (CGH, 17) |
| | | phosphatase 2A- | | | | |
| | | specific inhibitor | | | | |
| | | (I2PP2A) | | | | |
| EA58 | ARHE | Ras homolog gene | 2.2 | 0.0100 | 2q23.3 | |
| | NM_005168 | family, member E | | | | |

*Common chromosome aberrations previously identified in endometriosis tissue or cell line by CGH, FISH and R-binding (see, e.g., Gogusev et al, 1999 Hum. Genet., 105: 444-451; Gogusev et al, 2000 Mol. Hum. Reprod., 6: 821-827; Shin et al, 1997 Hum. Genet., 100: 401-406; Bouquet de Joliniere et al, 1997 Hum. Reprod. Update, 3: 117-123).

### EXAMPLE 7

### Clustering of Gene Expression

Cluster analysis was performed to identify the specific gene groups of interest and to display the global picture of differential regulation of 76 candidate genes in 15 pairs of clinical samples. The genes and clinical samples were grouped on the basis of similarities of gene expression patterns by hierarchical clustering algorithm using the Cluster software (Eisen et al, 1998 cited above). The result was displayed by TreeView software (Eisen et al, 1998 cited above) and shown in FIG. 5B. Based on the gene expression patterns, the 76 candidate genes were generally divided in four different groups, i.e. b1 , b2, b3 and b4 (FIG. 5B). The group b1 included the genes consistently over-expressed in most endometriosis samples while the genes in the group b3 were consistently under-expressed. The group b2 included most genes whose expression patterns exhibited a transition from over-expression to under-expression in endometriosis across FIG. 4B from the left to right. The group b4 included three immediate-early genes and one uncharacterized gene, showing a unique expression pattern that is totally different from those of the other 72 genes.

A zoom-in picture for the 14 best candidate genes (Table 3) were similarly displayed in FIG. 5C to demonstrate how these best candidate genes from the group b1 and b3 (FIG. 5B) were differentially regulated in each paired samples. It was clearly demonstrated that the 10 candidate genes on the top part in FIG. 5C were consistently over-expressed in the 15 cases while the other 4 genes on the bottom were consistently under-expressed.

### Example 8

### Demonstration of Clinical Co-relations

As shown in FIG. 5B, based on the similarities of gene expression patterns by hierarchical clustering, the majority of cases with early disease (stage I & II) clustered on the left-hand side of the figure, whereas the cases with advanced disease (stage III & IV) aggregated on the right-hand side. It is noteworthy that moving across FIG. 5B from the left to right, the expression patterns of the genes in the group b2 exhibited a transition from over-expression to under-expression in endometriosis when compared to the paired endometrium tissue, indicating that some genes may be differentially regulated at different stages of the disease. In order to identify the genes with stage-specific regulation, statistic analyses for 76 candidate genes were performed in two groups of patients, namely, early disease (stage I & II) and advanced disease (stage III & IV), respectively. Employing the same criteria of mean fold-change of 2.0 and statistically significant ΔΔC_{T} with P≦0.01, 45 genes were differentially expressed in the paired samples in at least one group of patients. Of these 45 genes, 34 were specific to the early disease, 4 specific for the advanced disease and the remaining 7 genes were differentially expressed in both groups. The detailed information was shown in Table 4.

TABLE 4
Statistic analysis of differential gene expression

| | | | | | | | |
|---|---|---|---|---|---|---|---|
| | | Mean fold | | Mean fold | | Mean fold | |
| cDNA | Sequence | change at | | change at stage | | change in all | |
| clone | homology | stage I & II | P value | III & IV | P value | (I-IV) | P value |

Overexpression in endometriosis

| | | | | | | | |
|---|---|---|---|---|---|---|---|
| EA30 | LOC151361 | 8.7 | 0.0108 | 4.2 | 0.0195 | 5.6 | 0.0003 |
| EA62 | PIM2 | 3.9 | 0.0009 | 3.1 | 0.0238 | 3.4 | 0.0002 |
| EA19 | RPL41 | 7.7 | 0.0015 | 1.8 | 0.1225 | 3.2 | 0.0017 |
| EA41 | PSAP | 4.9 | 0.0008 | 2.3 | 0.0030 | 3.1 | 0.0000 |
| EA29 | FBLN1 | 5.0 | 0.0055 | 2.1 | 0.0205 | 3.0 | 0.0003 |
| EA08 | Novel | 4.2 | 0.0001 | 2.0 | 0.0003 | 2.7 | 0.0000 |
| EA17 | HSPC157 | 4.9 | 0.0003 | 1.5 | 0.1549 | 2.4 | 0.0018 |
| EA20 | Novel | 4.0 | 0.0006 | 1.5 | 0.2263 | 2.2 | 0.0038 |
| EA53 | FLJ37272 | 4.8 | 0.0009 | 1.3 | 0.3178 | 2.2 | 0.0055 |
| EA44 | SIPL | 3.4 | 0.0012 | 1.4 | 0.2141 | 2.0 | 0.0041 |
| EA74 | FLJ22547 | 4.2 | 0.0040 | −1.2 | 0.7114 | 1.6 | 0.1743 |
| EA64 | NBP | 3.8 | 0.0008 | 1.2 | 0.6200 | 1.9 | 0.0283 |
| EA57 | ERH | 3.7 | 0.0041 | −1.1 | 0.7171 | 1.6 | 0.1605 |
| EA42 | FLJ12619 | 3.4 | 0.0020 | 1.2 | 0.3660 | 1.8 | 0.0085 |
| EA75 | FLJ33814 | 3.2 | 0.0058 | 1.0 | 0.9827 | 1.6 | 0.0740 |
| EA14 | HTR2A | 3.2 | 0.0021 | 1.1 | 0.6222 | 1.7 | 0.0183 |
| EA38 | FLJ10416 | 3.1 | 0.0009 | 1.3 | 0.0508 | 1.9 | 0.0007 |
| EA63 | MRG15 | 3.1 | 0.0033 | 1.3 | 0.2364 | 1.8 | 0.0053 |
| EA59 | SRRM1 | 3.1 | 0.0024 | 1.1 | 0.6553 | 1.7 | 0.0265 |
| EA21 | LOC126133 | 3.0 | 0.0035 | 1.1 | 0.6367 | 1.7 | 0.0242 |
| EA54 | HNRPL | 2.9 | 0.0024 | 1.0 | 0.9539 | 1.5 | 0.0577 |
| EA04 | MYBPC | 2.8 | 0.0000 | 1.4 | 0.2096 | 1.8 | 0.0030 |
| EA50 | CDK2 | 2.8 | 0.0041 | 1.3 | 0.4116 | 1.8 | 0.0233 |
| EA06 | NDUFA5 | 2.8 | 0.0025 | 1.3 | 0.1868 | 1.7 | 0.0034 |
| EA18 | TSAP19 | 2.8 | 0.0097 | 1.0 | 0.9409 | 1.5 | 0.1553 |
| EA73 | Novel | 2.7 | 0.0036 | 1.5 | 0.1061 | 1.9 | 0.0019 |
| EA15 | RPS25 | 2.4 | 0.0004 | 1.4 | 0.1348 | 1.7 | 0.0018 |
| EA36 | Adam17 | 2.4 | 0.0002 | 1.3 | 0.1679 | 1.7 | 0.0019 |
| EA25 | Ss18 | 2.4 | 0.0109 | 1.1 | 0.6457 | 1.5 | 0.0346 |
| EA11 | FLJ10952 | 2.2 | 0.0093 | 1.5 | 0.0107 | 1.7 | 0.0003 |
| EA56 | IMAGE: | 2.2 | 0.0126 | 1.0 | 0.8324 | 1.3 | 0.1317 |
| | 4856273 | | | | | | |
| EA70 | ARFRP1 | 2.2 | 0.0037 | −1.2 | 0.3232 | 1.2 | 0.2734 |
| EA69 | IMAGE: | 2.2 | 0.0043 | −1.3 | 0.0146 | 1.2 | 0.3469 |
| | 3860421 | | | | | | |
| EA37 | SPIN | 2.2 | 0.0257 | −1.5 | 0.0582 | 1.1 | 0.6988 |
| EA51 | FLJ11472 | 2.2 | 0.0165 | −1.5 | 0.1262 | 1.1 | 0.8099 |
| EA01 | CTNNB1 | 2.1 | 0.0108 | 1.1 | 0.6785 | 1.5 | 0.0786 |
| EA71 | IMAGE: | 2.1 | 0.0106 | −1.3 | 0.2105 | 1.1 | 0.5708 |
| | 3048642 | | | | | | |
| EA13 | KIAA0495 | 2.0 | 0.0040 | −1.1 | 0.6953 | 1.3 | 0.1230 |

Underexpression in endometriosis

| | | | | | | | |
|---|---|---|---|---|---|---|---|
| EA33 | DLX5 | −3.7 | 0.0129 | −38.7 | 0.0000 | −15.2 | 0.0000 |
| EA60 | HSD11B2 | −1.4 | 0.4760 | −5.6 | 0.0010 | −3.2 | 0.0023 |
| EA27 | SET | −3.0 | 0.0222 | −2.9 | 0.0331 | −2.9 | 0.0015 |
| EA58 | ARHE | −1.6 | 0.2675 | −2.7 | 0.0248 | −2.2 | 0.0100 |
| EA52 | COL7A1 | 2.0 | 0.0722 | −5.4 | 0.0030 | −2.1 | 0.0912 |
| EA16 | RPL10A | −1.3 | 0.2875 | −2.3 | 0.0092 | −1.8 | 0.0050 |
| EA22 | JUN-D | −3.3 | 0.0319 | −1.1 | 0.8064 | −1.7 | 0.1157 |

### Example 9

### Differential Regulation of Several Immediate-early Genes

As stated above, three of four genes in the group b4 (FIG. 5B) are known to be the immediate-early genes, including EA22 (jun-D), EA26 (Egr-1) and EA40 (c-jun). These three genes behaved very similarly in terms of the relative expression ratio in each pair of samples (b4, FIG. 5B). But they exhibited very distinct expression patterns in the 15 cases. Based on the expression data of these three genes, the 15 paired samples were re-grouped by clustering and displayed in FIG. 5D. It was clearly demonstrated that two opposite expression patterns, d1 and d3, and one transient state, d2, existed in these 15 cases. These three different patterns appear independent of the disease stages as well as the menstrual cycle (Table 2).

All publications cited in this specification are incorporated herein by reference herein. While the invention has been described with reference to a particularly preferred embodiment, it will be appreciated that modifications can be made without departing from the spirit of the invention. Such modifications are intended to fall within the scope of the appended claims.

1. A DNA molecule or genetic product thereof for monitoring the progression of, or diagnosing, or determining the severity of a gynaecological condition, the DNA molecule comprising a nucleotide sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, an equivalent thereof, a fragment thereof and combinations thereof.

2. The DNA molecule according to claim 1, which comprises SEQ ID NO: 8, an equivalent thereof, or a fragment thereof.

3. The DNA molecule according claim 1, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.

4. A DNA molecule or genetic product thereof, the DNA molecule comprising a nucleotide sequence selected from the group consisting of SEQ ID NO: 2, 6, 8, 11, 14, 17, 20, 24, 27, 28, 31, 34, 43, 44, an equivalent thereof, a fragment thereof, and combinations thereof.

5. A DNA molecule or genetic product thereof for monitoring the progression of, or diagnosing, or determining the severity of a gynaecological condition, the DNA molecule comprising a nucleotide sequence selected from the group consisting of GenBank accession numbers NM_{—}001904, AF126110, NM_{—}000094, NM_{—}001028, NM_{—}007104, NM_{—}021104, X51346, BC006226, NM_{—}006791, NM_{—}013293, AK094591, NM_{—}001533, NM_{—}005839, NM_{—}003011, NM_{—}020306, NM_{—}004450, BC018111, AF317228, NM_{—}001798, NM_{—}005168, NM_{—}003224, NM_{—}005000, AY007096, NM_{—}000196, NM_{—}025233, X84075, BC004275, NM_{—}018269, AK001814, XM_{—}031397, NM_{—}014179, NM_{—}152350, NM_{—}009280, L27560, AK001278, NM_{—}030939, AK021534, BC014498, BC011980, BC010281, AK026200, and AK091133, an equivalent thereof, a fragment thereof, and combinations thereof.

6. The DNA molecule according claim 5, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.

7. A method for monitoring the progression of, or diagnosing, or determining the severity of a gynaecological condition in a subject, the method comprising
determining the expression level of a gene comprising a nucleotide sequence according to claim 1 in the subject, and
comparing the expression level of the gene to the expression level of the same or a similar gene obtained from a reference sample, wherein a positive diagnosis is made if the expression level in the gene is statistically different to that found in the reference sample.

8. The method according to claim 7 wherein the expression level is determined by analysis of a gene transcript.

9. The method according to claim 8 wherein analysis of the transcript is performed by a method selected from the group consisting of Northern blot, quantitative PCR and sequencing

10. The method according to claim 7 wherein the expression level is determined by analysis of a protein encoded by the gene.

11. The method according to claim 10 wherein the analysis of the protein is performed by a method selected from the group consisting of Western blot, ELISA, surface plasmon resonance, and amino acid sequencing.

12. The method according to claim 7 wherein the expression levels of at least 5 genes are considered.

13. The method according to claim 7 wherein the expression levels of at least 10 genes are considered.

14. The method according claim 7, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.

15. A method for monitoring the progression of, or diagnosing, or determining the severity of a gynaecological condition, the method comprising the detection of a mutation in a gene, the mutation capable of producing a protein with a higher or lower biological activity than a protein from a non-mutated gene.

16. The method according claim 15, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.

17. A probe or primer for monitoring the progression of, or diagnosing, or determining the severity of a gynaecological condition that is capable of hybridising to a DNA molecule or genetic product thereof according to claim 1.

18. The probe or primer according to claim 17 comprising nucleotide sequences selected from the group consisting of SEQ ID NO: 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 214, 125, 126, 127, 128, 129, 30, 131, 132, 133, 134, and 135, an equivalent thereof, a fragment thereof, and combinations thereof.

19. The probe or primer according claim 17, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.

20. A kit for monitoring the progression of, or diagnosing, or determining the severity of a gynaecological condition comprising a probe or primer according to claim 17.

21. A method of screening for a compound having efficacy in the treatment or prevention of a gynaecological condition, the method including determining whether the candidate compound is capable of normalizing the expression levels of a gene comprising a DNA molecule according to claim 1.

22. A method of screening for a compound having efficacy in the treatment or prevention of a gynaecological condition, said method including determining whether the candidate compound is capable of acting as an agonist or antagonist to the protein product of a gene comprising a DNA molecule according to claim 1.

23. The method according to claim 22, wherein the gynaecological condition is selected from the group consisting of (i) a condition related to a disorder of uterine, fallopian, ovarian, cervical or vaginal tissue; (ii) the uncontrolled growth or ectopic growth of uterine tissue; and (iii) a condition selected from the group consisting of endometriosis, adenomyosis, and endometrioma.
